(19) United States
(12) Patent Application Publication
(10) Pub. No.: US 2026/0226353 A1
(43) Pub. Date: Aug. 6, 2026

(54) THERMAL ENERGY RECOVERY DEVICES FOR ELECTRICALLY HEATED CRACKING PROCESSES

(71) Applicants: Linde GmbH, Pullach (DE); BASF SE, Ludwigshafen am Rhein (DE); ARVOS GmbH, Kassel (DE); SABIC Global Technologies B.V., Bergen OP Zoom (NL)

(72) Inventors: Tobias SINN, Pullach (DE); Christopher EBERSTEIN, Pullach (DE); Mathieu ZELLHUBER, Pullach (DE); Gunther SCHMIDT, Pullach (DE); Jens HETZER, Kassel (DE); Andreas LINNENKOHL, Kassel (DE); Thorsten SCHLIETER, Kassel (DE); Karsten STÜCKRATH, Kassel (DE); Jörg WEIDENFELLER, Kassel (DE); Robert BROEKHUIS, Sugar Land, TX (US); Joseph William SCHROER, Sugar Land, TX (US); Raul VELASCO PELAEZ, Sugar Land, TX (US); Scott A. STEVENSON, Sugar Land, TX (US); Michael Edward HUCKMAN, Sugar Land, TX (US); Arno Johannes Maria OPRINS, Geleen (NL); Andrea HAUNERT, Ludwigshafen am Rhein (DE)

(21) Appl. No.: 19/158,383

(22) PCT Filed: Feb. 19, 2024
(86) PCT No.: PCT/EP2024/054129

§ 371 Date: Aug. 20, 2025

Related U.S. Application Data
(60) Provisional application No. 63/486,064, filed on Feb. 21, 2023.

(30) Foreign Application Priority Data
Mar. 17, 2023 (EP) 23162686.2

(51) Int. Cl. C10G 9/00; C07C 4/04; C10G 9/24; F28D 7/10; F28D 7/16

(52) U.S. Cl. CPC C10G 9/002; C07C 4/04; C10G 9/24; F28D 7/10; F28D 7/16; C10G 2300/1081; C10G 2400/20

(57) ABSTRACT

A method to produce olefins may include at least one cooling step in a thermal energy recovery by supplying a hydrocarbon feed to an outer tube of the thermal energy recovery assembly; heating the hydrocarbon feed in the outer tube of the thermal energy recovery assembly to output a preheated hydrocarbon feed; supplying the preheated hydrocarbon feed to an electrically powered cracking furnace comprising a reaction zone to heat the preheated hydrocarbon feed; cracking the preheated hydrocarbon feed in the reaction zone of the electrically heated cracking furnace using heat generated by electricity to output hot reactor effluent comprising cracked hydrocarbons and olefins; supplying the hot reactor effluent to an inner tube of the thermal energy recovery assembly; cooling the hot reactor effluent in the inner tube of the thermal energy recovery assembly by transferring heat to the hydrocarbon feed; and where the inner tube outside temperature in the thermal energy recovery assembly stays below 720° C. and the inner tube inside temperature in the thermal energy recovery assembly remains above 180° C.

### TECHNICAL FIELD

The present disclosure relates to systems and methods for transferring thermal energy and, more particularly, to energy recovery devices for transferring thermal energy from a hot effluent to a feed for a reactor furnace.

### BACKGROUND

Steam cracking of hydrocarbon feed in gas-fired steam cracking furnaces is the predominant commercial method for producing olefins. In such methods, hydrocarbons such as ethane, propane, butane, condensate, light naphtha, heavy naphtha, gas oil, pyrolysis oil, materials derived from processing refinery streams, Fischer-Tropsch products, plastic waste, or biofeedstocks are heated from roughly 550-650° C. to temperatures sometimes approaching 850° C. to facilitate conversion to light olefins, such as ethylene and propylene. Because cracking reactions are endothermic, significant amounts of heat must be provided.

To provide the energy required for conventional steam cracking processes, natural gas and/or light gas may be combusted in gas-fired steam cracking furnaces. Combustion of the hydrocarbons in the gas-fired steam cracking furnace forms carbon dioxide, which is emitted as part of flue gas from the gas-fired steam cracking furnace. Such emissions may be undesirable in view of current environmental considerations. Olefins are a major chemical building block and may often be produced in large quantities, from several hundred thousand tons per year in a small cracker to two million tons per year or more at a single large olefin production facility. As a result, the production of olefins using gas-fired steam cracking furnaces may result in an undesirably high emission of carbon dioxide.

In a conventional gas-fired olefins production furnace, the combustion gases can only supply heat to the cracking reaction when the temperature exceeds the temperature of reaction, for example, from 550-650° C. to 850° C. Once the gases have cooled below this temperature, there may be a desire to extract as much of the remaining heat as possible to achieve energy-efficient operation of the plant. This heat is conventionally recovered in a so-called convection section. Often the energy is used to preheat the reactor feeds and diluent steam to the temperature needed for the cracking reaction to occur.

In olefin production, once the feed is cracked, the reactor effluent should be cooled before further processing of the cracked gas. Ideally, the initial cooling occurs quickly to reduce or prevent side reactions in the reactor effluent while it is still at relatively high temperatures. In addition, for an energy-efficient process, the heat of the reactor effluent should be recovered to the greatest extent possible and used elsewhere in the process.

In some systems, this quenching and cooling takes place in transfer line exchangers (TLE), where the reactor effluent is cooled by exchanging heat with liquid water to produce high-pressure steam. Cooling by exchanging heat with boiling water has the advantage that heat transfer is generally more rapid than when cooling against a gas, sometimes five or even ten times faster for the same exchanger geometry. This steam may be used to power steam turbines or other auxiliaries. For example, the steam is commonly used to power the cracked gas compressor, one or more of the refrigeration compressors, or one or more pumps. The use of steam to drive rotating equipment such as compressors and pumps is a convenient way to use the energy recovered from cooling the reactor effluent, but the efficiency of converting energy in the form of heat (e.g., the heat contained in steam) to mechanical work is typically low, in the range of 30 to 50%.

One solution to reducing the large amount of carbon dioxide produced by conventional steam cracking processes would be electrified steam cracking. Electrified steam cracking includes the use of cracking furnaces that are at least in part directly or indirectly heated by electricity. Electrified steam cracking furnaces have reduced emissions as compared to gas-fired cracking furnaces.

However, electrified steam cracking furnaces introduce new technical challenges that must be overcome. One consequence of using an electrified steam cracking furnace is the elimination of hot flue gas from the combustion of fuel in the gas-fired cracking furnace, so that heating typically supplied by the hot flue gas, including feed preheating, must be supplied another way. A second consequence is the need for different energy integration within the cracking process. Rotating equipment such as compressors and pumps are easily powered by electricity; moreover, the efficiency of operation of such equipment with electricity is much higher than with steam, with energy efficiencies of greater than 90% commonly achieved when using electricity as compared to 30 to 50% typically obtained when using steam. Therefore, in an electric steam cracking process there is considerable incentive to power these devices using electricity. This means that the energy currently recovered when cooling the reactor effluent can no longer be used for powering pumps and compressors; instead there is a need to find a different use for this energy so that the overall energy efficiency of the process can be maintained at a high level.

For electrically powered processes, Applicant has identified a need for systems and methods for using the energy obtained from quenching hot reactor effluent while still quenching the cracked gas quickly enough to prevent further reaction. Applicant has also identified a need for systems and methods for preheating a feed for an electrically powered furnace.

### SUMMARY

In an embodiment of the present disclosure, a method to produce olefins may include at least one cooling step in a thermal energy recovery assembly. The cooling step may be characterized by supplying a hydrocarbon feed to an outer tube of the thermal energy recovery assembly; heating the hydrocarbon feed in the outer tube of the thermal energy recovery assembly to output a preheated hydrocarbon feed; supplying the preheated hydrocarbon feed to an electrically powered cracking furnace comprising a reaction zone to heat the preheated hydrocarbon feed; cracking the preheated hydrocarbon feed in the reaction zone of the electrically heated cracking furnace using heat generated by electricity to output hot reactor effluent comprising cracked hydrocarbons and olefins; supplying the hot reactor effluent to an inner tube of the thermal energy recovery assembly; and cooling the hot reactor effluent in the inner tube of the thermal energy recovery assembly by transferring heat to the hydrocarbon feed. The inner tube outside temperature in the thermal energy recovery assembly may stay below 720° C. and the inner tube inside temperature in the thermal energy recovery assembly may remain above 160° C., preferably above 180° C.

In certain embodiments, the inner tube outside temperature may stay below 720° C. for operation with ethane type feedstock, stays below 700° C. for operation with propane type feedstock, may stay below 680° C. for operation with butane type feedstock, may stay below 660 for operation with naphtha type feedstock, and may stay below 640° C. for operation with feedstocks heavier than naphtha. In certain embodiments, the inner tube inside temperature may stay above 160° C. for operation with ethane type feedstock, may stay above 170° C. for operation with propane type feedstock, may stay above 180° C. for operation with butane type feedstock, and may stay above 270° C. for operation with naphtha type or heavier feedstocks.

In certain embodiments, the hot reactor effluent may be first partially cooled against the hydrocarbon feed through a co-current section and then further cooled against the hydrocarbon feed in a countercurrent section.

In certain embodiments, the hot reactor effluent is first cooled against the hydrocarbon feed through a co-current section, further cooled through a steam generating section, and then further cooled against the hydrocarbon feed in a counter-current section.

In certain embodiments, the hydrocarbon feed passes first through the co-current section, and then through the countercurrent section. In certain embodiments, the hydrocarbon feed passes first through the countercurrent section, and then through the co-current section. In certain embodiments, the countercurrent section is a separate piece of equipment and is a tube-in-tube or shell-and-tube type heat exchanger.

In certain embodiments, a thermal energy recovery assembly may include an outer tube; and an inner tube; wherein a hydrocarbon feed is supplied to an electrically powered cracking furnace comprising a reaction zone to heat the hydrocarbon feed; wherein the inner tube comprises a first inlet configured to receive the hot reactor effluent from the electrically powered cracking furnace; and wherein the outer tube is disposed about the inner tube to enclose an annulus about the inner tube, the annulus comprising a second inlet configured to receive the hydrocarbon feed.

In certain embodiments, the annulus comprises at least one heat transfer enhancement to enhance heat transfer from the inner tube to the annulus. At least one heat transfer enhancement may include one or more of impingement, turbulence promotion, high-shear-inducing geometry, or increased surface area. The annulus may include a plate impingement between an upstream end and a downstream end, the plate impingement including; a first channel having a stage inlet at the upstream end and being closed to flow at the downstream end; a second channel having a stage outlet at the downstream end, the second channel being disposed between the first channel and the inner tube; a wall separating the first channel from the second channel, the wall defining openings to fluidly connect the first channel and the second channel; and the plate impingement being configured to receive feed through the stage inlet, flow feed from the first channel to the second channel via the openings of the wall, and causing flow of the feed to impinge onto an outer surface of the inner tube and exhaust feed through the stage outlet of the second channel. The annulus may include a piccolo impingement, the piccolo impingement including an upstream divider disposed about the inner tube and within the outer tube; a downstream divider disposed about the inner tube and within the outer tube downstream of the upstream divider within the annulus, the downstream divider defining at least one stage outlet; a chamber defined within the outer tube and about the inner tube between the upstream divider and the downstream divider; and piccolo tubes offset from the inner tube, the piccolo tubes extending through the chamber from the upstream divider to the downstream divider, the piccolo tubes including a stage inlet for receiving incoming feed, the piccolo tubes including a plurality of openings defined therein, the piccolo impingement being configured to receive feed from the stage inlets, flow feed from the piccolo tubes into the chamber via the plurality of openings, and exhaust feed from the chamber via the at least one stage outlet.

In certain embodiments, the thermal energy recovery assembly may include a plurality of inner tubes parallel with one another with each inner tube disposed within an outer tube, each outer tube having one or more of impingement, turbulence promotion, high-shear-inducing geometry, or increased surface area to enhance heat transfer from the inner tube to an annulus defined within the outer tube.

Certain embodiments may include a use of a thermal energy recovery assembly for conducting the method to produce olefins

Still other aspects and advantages of these exemplary embodiments and other embodiments, are discussed in detail herein. Moreover, it is to be understood that both the foregoing information and the following detailed description provide merely illustrative examples of various aspects and embodiments, and are intended to provide an overview or framework for understanding the nature and character of the claimed aspects and embodiments. Accordingly, these and other objects, along with advantages and features of the present disclosure, will become apparent through reference to the following description and the accompanying drawings. Furthermore, it is to be understood that the features of the various embodiments described herein are not mutually exclusive and may exist in various combinations and permutations.

### BRIEF DESCRIPTION OF THE DRAWINGS

The accompanying drawings, which are included to provide a further understanding of the embodiments of the present disclosure, are incorporated in and constitute a part of this specification, illustrate embodiments of the present disclosure, and together with the detailed description, serve to explain principles of the embodiments discussed herein. No attempt is made to show structural details of this disclosure in more detail than may be necessary for a fundamental understanding of the embodiments discussed herein and the various ways in which they may be practiced. According to common practice, the various features of the drawings discussed below are not necessarily drawn to scale. Dimensions of various features and elements in the drawings may be expanded or reduced to more clearly illustrate embodiments of the disclosure.

FIG. 1 is a schematic view of a portion of an example furnace assembly for heating a feed to provide a hot reactor effluent in accordance with embodiments of the present disclosure;

FIG. 2A is a partial schematic section side view of an example thermal energy recovery assembly according to embodiments of the present disclosure;

FIG. 2B is a partial schematic section end view taken along line B-B of the example thermal energy recovery assembly shown in FIG. 2A according to embodiments of the present disclosure;

FIG. 3A is a partial schematic perspective view of another example thermal energy recovery assembly according to embodiments of the disclosure;

FIG. 3B is a partial schematic section end view taken along line B-B of the example thermal energy recovery assembly shown in FIG. 3A according to embodiments of the present disclosure;

FIG. 4A is a schematic section view of an example inner tube including example rounded projections on an interior surface of the inner tube according to embodiments of the disclosure;

FIG. 4B is a schematic section view of another example inner tube including example rectangular projections on an interior surface of the inner tube according to embodiments of the disclosure;

FIG. 5 is a block diagram of an example method to produce olefins according to embodiments of the disclosure.

FIG. 6A is a block diagram of a single-section embodiment of an exemplary feed-effluent exchanger.

FIG. 6B is a block diagram of a two-section embodiment of an exemplary feed-effluent exchanger.

### DETAILED DESCRIPTION

The drawings include like numerals to indicate like parts throughout the several views, the following description is provided as an enabling teaching of exemplary embodiments, and those skilled in the relevant art will recognize that many changes may be made to the embodiments described. It also will be apparent that some of the desired benefits of the embodiments described may be obtained by selecting some of the features of the embodiments without utilizing other features. Accordingly, those skilled in the art will recognize that many modifications and adaptations to the embodiments described are possible and may even be desirable in certain circumstances. Thus, the following description is provided as illustrative of the principles of the embodiments and not in limitation thereof.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to,” unless otherwise stated. Thus, the use of such terms is meant to encompass the items listed thereafter, and equivalents thereof, as well as additional items. The transitional phrases “consisting of” and “consisting essentially of,” are closed or semi-closed transitional phrases, respectively, with respect to any claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish claim elements.

In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

Applicant has identified preferred operating windows/regimes to prevent the thermal energy recovery assembly equipment from undesirable performance loss through feed pre-cracking and/or effluent coking mechanisms.

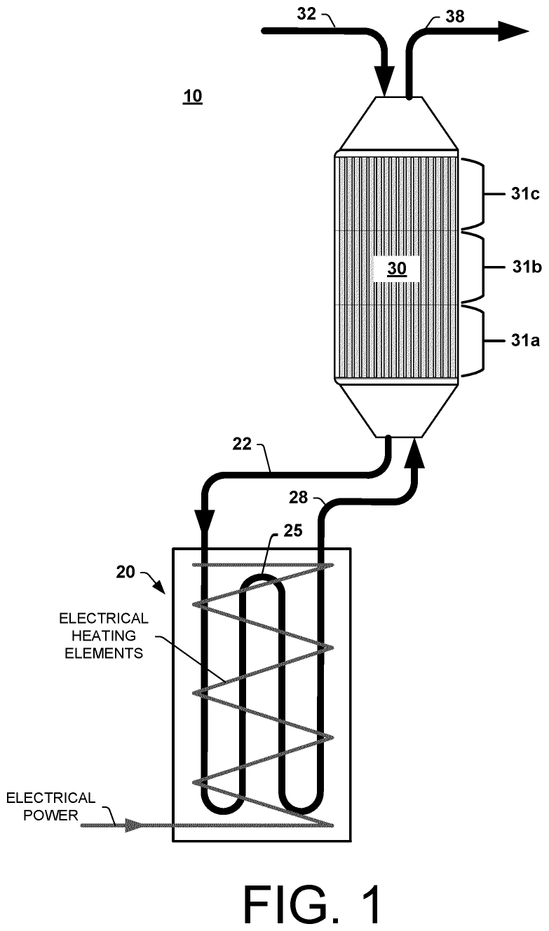

FIG. 1 schematically illustrates an example furnace assembly 10 for heating a feed to provide a hot reactor effluent according to embodiments of the disclosure. In some embodiments, the furnace assembly 10 may be used for producing olefins from hydrocarbons. As shown in FIG. 1, the furnace assembly 10 may include an electrically powered furnace 20 and a thermal energy recovery assembly 30. As used herein, the phrase “hot reactor effluent” refers to reactor effluent that is downstream of the furnace 20 and is being cooled from the temperature at which reactor effluent exited the furnace 20. In some embodiments, the thermal energy recovery assembly 30 may include one or more stages, for example, a first stage 31a, a second stage 31b, and a third stage 31c, for example, as shown in FIG. 1. As used herein, the term stage refers to a discrete part of the thermal energy recovery assembly that is configured to bring the feed into indirect thermal contact with the hot reactor effluent, e.g., using heat transfer enhancement methods described in more detail below. In some embodiments including more than one stage, the stages may have substantially the same structural configuration, and in some embodiments including more than one stage, one or more of the stages may have a structural configuration that differs from the structural configuration of other stages. In some embodiments including more than one stage, the thermal energy recovery assembly may include two or more sections, each section including one or more stages. Within a section that includes more than one stage, feed flows through these stages sequentially, defining an overall flow direction that may be either co-current or countercurrent to the direction of the flow of hot reactor effluent. Each section may have a corresponding inlet and outlet for feed, and may process either the same or a different (portion of) feed. Some sections may have co-current configuration while others have countercurrent configuration, within the same thermal energy recovery assembly. When the thermal energy recovery assembly has more than one section, the sections may be implemented in a single piece of equipment, or in different pieces of equipment. For example, the first section may be implemented as a tube-in-tube exchanger to heat hydrocarbon feed with heat transfer enhancements as described in more detail below, the second section may be implemented as a steam-generating transfer line exchanger, and the third section may be implemented as a conventional countercurrent exchanger to heat hydrocarbon feed. As used herein, meaning of the words upstream and downstream shall be relative to the flow direction of the hot reactor effluent, i.e. if a first section performs cooling of the hot reactor effluent, and a second section performs further cooling of the partially cooled reactor effluent, the second section is downstream of the first section, regardless of the configuration and direction of the coolant streams. As used herein, co-current and countercurrent shall be relative to the flow direction of the hot reactor effluent, i.e. a section in which the overall flow direction of the coolant is in the opposite direction of the hot reactor effluent shall be considered a countercurrent section.

In some embodiments, the furnace 20 may be configured to receive a feed and heat the feed to a reaction temperature to provide a hot reactor effluent. In some embodiments, the feed and/or the effluent may be in the form of a liquid, a gas, or a combination thereof. For example, the furnace 20 may be an electrically powered cracking furnace and the feed may be, or include, hydrocarbons for cracking in the furnace 20 to provide a hot reactor effluent including cracked hydrocarbons, for example, in an at least partially gaseous state (e.g., a completely gaseous state). In some embodiments, the furnace 20 may be configured to heat the feed to a cracking temperature to break the hydrocarbons into desired products that may be exhausted from the furnace 20 as a hot reactor effluent. The feed may include, for example, ethane, propane, butane, condensate, light naphtha, heavy naphtha, gas oil, pyrolysis oil, materials derived from processing refinery streams, Fischer-Tropsch products, plastic waste, and/or biofeedstocks. The feed may additionally include steam. In some embodiments, the furnace 20 may be configured to receive the feed into one or more reactor zones or reactor chambers 25 (for example, a cracker tube or a cracking coil) via a reactor feed line 22 and exhaust the hot reactor effluent via a reactor effluent line 28. In some embodiments, the furnace 20 may be electrically heated to a cracking temperature, for example, such that the feed substantially continuously flows into the reactor chamber(s) 25 via the reactor feed line 22 and out of the reactor chamber(s) 25 as hot reactor effluent via the reactor effluent line 28. The reactor chamber(s) 25 may be heated directly or indirectly by electrical power. To increase the efficiency of the furnace 20, the feed may be preheated to a temperature closer to the cracking temperature of the feed prior to (e.g., upstream relative to) entering the reactor chamber(s) 25. Reactor feed line 22 and reactor effluent line 28 may be configured to traverse additional equipment, for example heat transfer equipment, not depicted in FIG. 1.

The thermal energy recovery assembly 30, in some embodiments, may be, or include, a gas-to-gas energy recovery device or heat exchanger. The thermal energy recovery assembly 30 may be configured to receive the hot reactor effluent from the reactor chamber(s) 25 and quench the hot reactor effluent to a quenched temperature to preserve the desired products within the reactor effluent and/or to prevent side reactions from occurring within the reactor effluent, for example, as the hot reactor effluent cools. In some embodiments, a single thermal energy recovery assembly 30 may receive hot reactor effluent from multiple reactor chambers 25. In certain embodiments, a single reactor chamber 25 may provide hot reactor effluent to multiple thermal energy recovery assemblies 30. The ratio between the number of reactor chambers 25 and the number of thermal energy recover assemblies 30 may be in a range of 0.1 to 10, for example, a range of 0.5 to 2.

The hot reactor effluent enters the thermal energy recovery assembly 30 at a temperature of at least 550° C., at least 575° C., at least 600° C., at least 610° C., at least 620° C., at least 625° C., at least 630° C., at least 640° C., at least 650° C., at least 700° C., at least 750° C., at least 800° C., or at least 850° C. To quench the hot reactor effluent, the thermal energy recovery assembly 30 may utilize the feed as a cooling medium, for example, prior to the feed entering the reactor chamber 25 (e.g., upstream of the reactor chamber 25). As a result of cooling the hot reactor effluent within the thermal energy recovery assembly 30, the feed may be preheated to a temperature closer to the cracking temperature thereof (e.g., to a temperature of at least 350° C., at least 375° C., at least 400° C., at least 450° C., at least 500° C., or at least 550° C.). In some embodiments, the thermal energy recovery assembly 30 may be configured to preheat the feed over a cracking temperature, for example, such that cracking begins to occur in the thermal energy recovery assembly 30, for example, when the feed is preheated to a temperature greater than 550-650° C. As used herein, “quenched reactor effluent” refers to reactor effluent that has passed through the thermal energy recovery assembly 30. In some embodiments, the hot reactor effluent may additionally be partially cooled or quenched before or after passing through the thermal energy recovery assembly 30. In some embodiments, the preheated feed may be further heated (for example, by means of a fired heater or an electrical heater) before entering the reactor chamber.

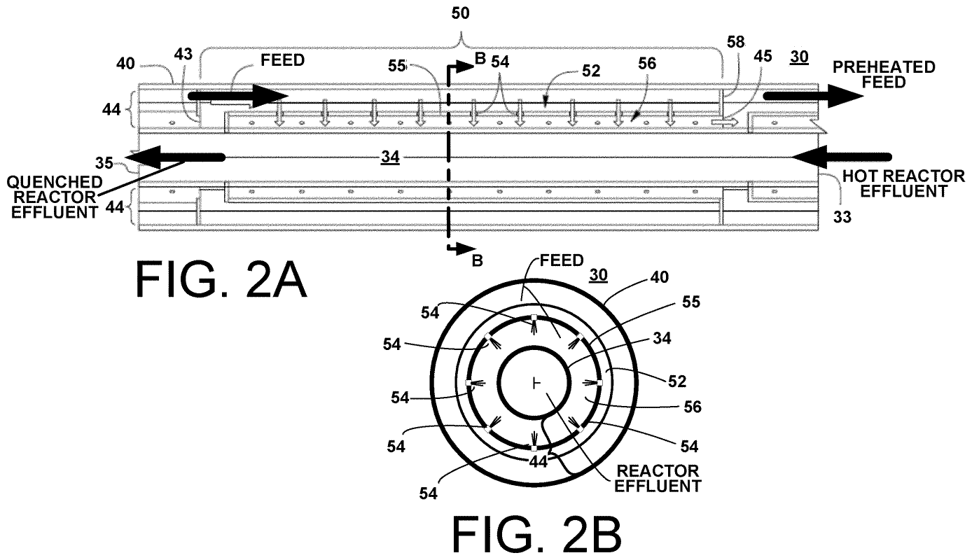

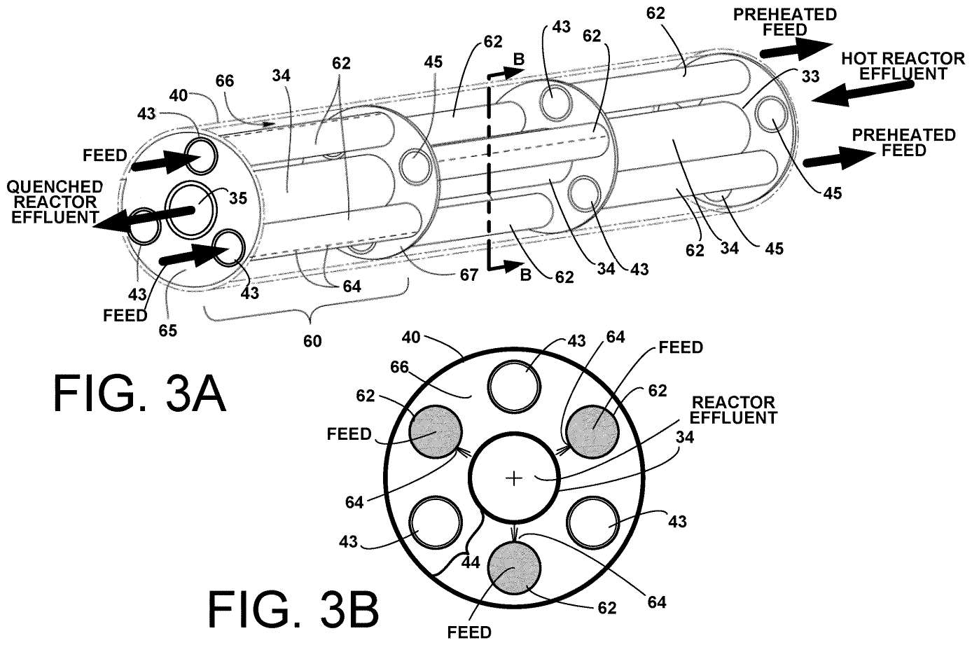

In some embodiments, a thermal energy recovery assembly 30 may be configured to recover thermal energy from hot reactor effluent to heat a feed to an electrically-powered reactor furnace. As explained herein with respect to FIG. 2A through FIG. 3B, in some embodiments, the thermal energy recovery assembly 30 may include an inner tube 34 and an outer tube 40. The inner tube 34 may include a first inlet that is configured to receive hot reactor effluent from an electrically-powered reactor furnace. The outer tube 40 may be disposed about the inner tube 34 to enclose an annulus 44 about the inner tube 34. Although the terms “annulus” and “annular” (and derivations therefrom) are used herein, the “annulus 44” may, or may not, be defined by inner and outer circles to result in an annular cross-section having inner and outer circular boundaries. In some embodiments, the inner and/or outer boundaries of the cross-section may have shapes other than circular, such as triangular, rectangular, polygonal, elliptical, oval-shaped, etc. In some embodiments, the central axis of the inner tube may coincide with the central axis of the outer tube. In some embodiments, the central axis of the inner tube may be offset from the central axis of the outer tube. The term “annular” (and derivations thereof) may be interpreted similarly. The annulus 44 may include a second inlet that is configured to receive a feed to the electrically-powered reactor furnace. The annulus 44 may be configured to use the feed for the electrically-powered reactor furnace as a cooling medium to recover thermal energy from the hot reactor effluent prior to the feed being supplied to the electrically-powered reactor furnace. The annulus 44 may be configured to enhance heat transfer from the hot reactor effluent to the feed. In some embodiments, hot reactor effluent may reach a first inlet via an effluent gas inlet chamber or other connector. In some embodiments, cooling may be supplied to a feed gas inlet chamber or other connector. In some embodiments, the effluent gas inlet chamber may connect one or more than one reaction chambers to one or more than one inner tube. In some embodiments, a header may be provided to connect the feed to more than one annulus 44. In some embodiments, cooled cracked gas from more than one inner tube 34 may be collected using a header. In some embodiments, heated feed from more than one annulus 44 may be combined via a header. In some embodiments, multiple annuli 44 may be contained in a single mechanical device, which may receive hot effluent from multiple cracking coils via a gas inlet chamber or other connector, and cold feed from a feed header.

In some applications, the residence time and/or the pressure drop of the reactor effluent in the thermal energy recovery assembly 30 may be influential on the process and/or the products achieved by the heating process in the furnace assembly 10. Both the residence time and the pressure drop occurring as the hot reactor effluent passes through the thermal energy recovery assembly 30 may be influential on ethylene selectivity of products produced by the furnace assembly 10. The residence time may be defined as the time that the hot reactor effluent exceeds a temperature greater than its cracking temperature, for example, greater than 550-650° C. In some embodiments, both the residence time and the pressure drop may be balanced during the quenching of the hot reactor effluent, for example, to preserve the ethylene selectivity of the reactor effluent. For example, an increased pressure drop in the thermal energy recovery assembly can affect the selectivity as a result of an increased pressure in cracking coils of the furnace assembly 10 which changes selectivity of the cracking reaction in the furnace assembly 10. With respect to residence time, a longer residence time may allow for additional side reactions in the thermal energy recovery assembly 30.

In some embodiments, the thermal energy recovery assembly 30 may be configured to quench the hot reactor effluent received from the furnace 20 using the feed to the furnace 20 as a cooling medium for the hot reactor effluent, for example, and such that the feed is preheated by the hot reactor effluent prior to entering the reactor chamber 25. For example, as shown in FIG. 1, the thermal energy recovery assembly 30 may receive the feed through a cold feed line 32 and provide preheated feed to the reaction chamber 25 through the reactor feed line 22. The thermal energy recovery assembly 30 may receive hot reactor effluent from the reactor effluent line 28 and provide quenched reactor effluent to a quenched effluent line 38.

In some embodiments, the thermal energy recovery assembly 30 may be configured to operate as a gas-to-gas heat exchanger to exchange heat from the hot reactor effluent to the feed. As a result of heat being exchanged between gases, it is more difficult to quench the reactor effluent within a desired residence time comparable to liquid-to-gas heat exchangers (e.g., steam-generating heat exchangers using boiling water as a relatively low-temperature cooling medium, typically used with gas-fired cracking furnaces), due, for example, to the generally lower heat transfer coefficients in gas-to-gas exchangers and the lower temperature difference between the hot and cold fluids. Therefore, additional design features for thermal energy recovery assembly 30 may be desired, as described in some of the exemplary embodiments below.

With reference to FIG. 2A and FIG. 2B, a portion of an example thermal energy recovery assembly 30 is shown having a tube-in-tube design with a hot reactor effluent flowing through an inner tube 34 (e.g., a central tube), and the feed flowing through an annulus 44 at least partially defined by the inner tube 34 and an outer tube 40. As shown, the inner tube 34 is disposed about a central axis of the thermal energy recovery assembly 30. In some embodiments, the inner tube 34 may be disposed about the central axis or offset from the central axis of the thermal energy recovery assembly 30.

The thermal energy recovery assembly 30 may be a co-current heat exchanger or counter-current heat exchanger (e.g., as shown) with the hot reactor effluent flowing through the thermal energy recovery assembly 30 in a first direction and the feed flowing through the thermal energy recovery assembly 30 in a second direction opposite the first direction. In some embodiments, the inner tube 34 may include an inlet 33 and an outlet 35, with the hot reactor effluent entering through the inlet 33, flowing through the inner tube 34, and exiting through the outlet 35. The annulus 44 may include an inlet 43 and an outlet 45, with the feed entering through the inlet 43, flowing through the annulus 44, and exiting through the outlet 45 as preheated feed. In such embodiments, the hot reactor effluent, at its highest temperature prior to being cooled by heat transfer to the feed to preheat the feed, enters the thermal energy recovery assembly 30 at the inlet 33 at a point adjacent to where the feed, at its highest temperature after being heated by the reactor effluent, exits the thermal energy recovery assembly 30 through the outlet 45 as preheated feed. The reactor effluent (e.g., a quenched reactor effluent), at its lowest temperature after heating the feed, exits the thermal energy recovery assembly 30 at the outlet 35 as quenched reactor effluent at a point adjacent to where the feed, at its lowest temperature prior to being heated by the hot reactor effluent, enters the thermal energy recovery assembly 30 through the inlet 43. In some such embodiments, the hot reactor effluent enters the thermal energy recovery assembly 30 at its highest temperature at the point where the feed exits the thermal energy recovery assembly 30 at its highest temperature, and the reactor effluent exits the thermal energy recovery assembly 30 at its lowest temperature where the feed enters the thermal energy recovery assembly 30 at its lowest temperature. In some embodiments, the thermal energy recovery assembly 30 may be a co-current heat exchanger with the reactor effluent and the feed flowing in the same direction within the thermal energy recovery assembly 30. In some embodiments, if the thermal energy recovery assembly 30 is made up of more than one section, some sections may be co-current and others may be counter-current.

In some embodiments, the inner tube 34 may be or include a bare or plain tube with a smooth inner surface. In some embodiments, the inner tube 34 may include heat transfer enhancements that promote turbulence or increase a surface area of the inner tube 34. For example, the inner tube 34 may include velocity rods or other turbulence-promoting structures. In some embodiments, the inner tube 34 may include fins (for example, straight and/or rifled fins, rectangular and/or rounded in cross-section) or other surfaces to increase a surface area in contact with the reactor effluent flowing through the inner tube 34. Such heat transfer enhancements within the inner tube 34 may decrease the residence time of the reactor effluent. The heat transfer enhancements may increase a pressure drop within the reactor effluent. In some embodiments, the inclusion of heat transfer enhancements within the inner tube 34 may be balanced, for example, to reduce pressure drop that may result from heat transfer enhancements. In certain embodiments, fouling may be expected such that frequent cleaning of the inner tube 34 may be required. In such embodiments, the inner tube 34 may be straight or bare to aid in cleaning. In particular embodiments, sections of the inner tube 34 may be bare, and sections of the inner tube 34 may include heat transfer enhancements, such as the turbulence-promoting structures and/or the area-increasing features mentioned above.

In some embodiments, the annulus 44 may include turbulence-promoting structures, such as, for example, winglets, artificial roughness, washboard/grooves, pin fins, and/or dimples. Such structures may increase the rate of heat transfer from the outer surface of the inner tube 34, and/or may increase the pressure drop of the feed traversing the annulus 44. Such structures may be used by themselves as a heat transfer enhancement, or may be used in combination with other heat transfer enhancements, such as, for example, the plate and piccolo impingements described herein.

In some embodiments, the annulus 44 may include a high-shear-inducing geometry configured to promote high-shear flow, for example, as may result from feed flowing at high velocity, such as greater than 50 meters per second (m/s), or greater than 60 m/s, or greater than 70 m/s, or greater than 80 m/s. In some embodiments, the direction of the high-shear feed flow through the annulus is substantially parallel to the inner tube. In some embodiments, the high-shear-inducing geometry may include configuring the outer tube such that the spacing between the outside surface of the inner tube 34 and the inside surface of the outer tube 40 is 10 millimeters (mm) or less, 8 mm or less, 6 mm or less, or 4 mm or less. The high shear rate may serve as a heat transfer enhancement by promoting a high rate of heat transfer from the flowing feed to the outside surface of the inner tube 34.

In some embodiments, heat transfer enhancement by impingement may refer to flow of fluid passing through the outer tube, whose average direction as it proceeds from inlet to outlet may be substantially parallel to the inner tube, being purposely directed to flow toward the inner tube, for example, using geometric features introduced into the annulus. In some embodiments, this directed (impinging) flow may, for example, be perpendicular to the inner tube, or directed toward the inner tube at an angle greater than thirty degrees relative to an axis of the inner tube, while its velocity may be relatively greater than a superficial velocity of the outer-tube fluid (for example, the volumetric flow of the outer-tube fluid divided by the area of the annular cross-section between the inner and outer tube). In some embodiments, the geometric features that promote impingement may include, for example, nozzles and/or openings oriented toward the inner tube, and/or obstructions placed in a flow path that may redirect fluid from a direction that is more parallel to the inner tube more directly towards the outer surface of the inner tube. These example features may be implemented in a periodic fashion, for example, resulting in impingement zones that occur at intervals along the length and/or the circumference of the inner tube. Applicant has discovered that introduction of such impingement features may increase the rate of heat transfer relative to the rate that would be attained by parallel flow through the outer tube. Moreover, Applicant has discovered that for a suitable level of heat transfer enhancement, a ratio of a velocity of an impinging flow to the superficial velocity may be greater than two, greater than five, or greater than ten. The velocity of the impinging flow may be approximated, in the case of nozzles or openings, as the volumetric flow divided by the total flow area defined by the nozzles or openings, through which the flow is directed. Additionally, the heat transfer enhancement may be found to be more suitable at distances between the impingement-inducing feature (e.g., between nozzles or openings 54) and the inner tube that are from about the diameter to about twelve times the diameter of the nozzles or openings 54, from about the diameter to about ten times the diameter, or from about two times the diameter to about eight times the diameter. Examples of impingement features may include plate impingement and/or piccolo impingement. In certain embodiments at least one heat transfer enhancement in a first stage may be the same or different from at least one heat transfer enhancement in a second stage.

Referring now to FIG. 2A, FIG. 2B, FIG. 3A, and FIG. 3B the annulus 44 (see FIGS. 2A and 2B) of the thermal energy recovery assembly 30 may include one more structures to promote heat transfer from the reactor effluent in the inner tube 34 to the feed in the annulus 44 of the thermal energy recovery assembly 30. For example, the thermal energy recovery assembly 30 may include plate impingements 50 in the annulus 44 of the thermal energy recovery assembly 30, for example, as shown in FIGS. 2A and 2B, and/or the thermal energy recovery assembly 30 may include piccolo impingements 60 (see also FIG. 3A) in the annulus 44 (see also FIG. 3B) of the thermal energy recovery assembly 30.

With particular reference to FIGS. 2A and 2B, the plate impingement 50 may include the cooling medium (e.g., the feed) entering a first channel 52 via a stage inlet 43 that may be spaced apart from the inner tube 34, for example, on an outside or outer circumference of the annulus 44 of the thermal energy recovery assembly 30. The cooling medium exits the first channel via one or more nozzles or openings 54 in a wall 55 into a second channel 56 that is in contact with the inner tube 34. The first channel 52 may terminate in a downstream end 58, for example, such that the cooling medium may be forced into the second channel 56 to flow through the annulus 44 and exit through a stage outlet 45 at a downstream end of the second channel 56. The wall 55 separates the first channel 52 from the second channel 56. The thermal energy recovery assembly 30 may include one or more plate impingements 50 disposed along the length thereof. Each plate impingement 50 as shown in FIGS. 2A and 2B may be considered a plate impingement stage with the thermal energy recovery assembly 30 including one or more plate impingement stages in series or parallel with one another.

In some embodiments, one or more of the nozzles or openings 54 may have a circular cross-section. In some such embodiments, the diameter of the one or more nozzles or openings 54 may range from about 1 millimeter (mm) to about 15 mm, for example, from about 2 mm to about 10 mm, from about 3 mm to about 8 mm, or from about 4 mm to about 7 mm. In nozzles or openings 54 not having a circular cross-section, the area of the cross-section of the nozzles or openings 54 may substantially correspond to the area of nozzles or openings 54 having a circular cross-section. In some embodiments, the nozzles or openings 54 may be circumferentially aligned at different points along the longitudinal length of the wall 55 or they may be circumferentially staggered along the longitudinal length of the wall, for example, in a helically extending manner. In some embodiments, wall 55 may be spaced from the outer surface of the inner tube 34 by a distance ranging, for example, when the nozzles or openings 54 have a circular cross-section, from about a distance equal to the diameter of the nozzles or openings 54 to about twelve times the diameter of the nozzles or openings 54, or from about the diameter to about ten times the diameter, from about two times the diameter to about eight times the diameter.

In some embodiments, the nozzles or openings 54 may be spaced around the circumference of the wall 55. For example, at a given point along the longitudinal length of the wall 55, the wall may include, for example, from one to fifteen nozzles or openings 54, which may at least partially depend on the dimensions of the inner tube 34, for example, with relatively more nozzles or openings 54 for relatively larger inner tubes 34. In some embodiments, the nozzles or openings 54 may be circumferentially spaced around the inner tube 34, for example, such that the spacing equals pi (i.e., 3.14159) multiplied by the sum of the diameter of the outer surface of the inner tube 34 and two times the distance from the nozzles or openings 54 to the outer surface of the inner tube 34, all of which is divided by the number of nozzles or openings 54 around the circumference. In some embodiments, the nozzles or openings 54 may be spaced from one another substantially equally along the longitudinal length of the wall 55 and/or substantially equally circumferentially around the wall 55.

With reference to FIGS. 3A and 3B, the piccolo impingement 60 may include the cooling medium (e.g., the feed) entering one or more piccolo or outer tubes 62 and a chamber 66 defined about the inner tube 34. In some embodiments, the chamber 66 may generally define an annulus, such as, for example, the annulus 44 shown in FIGS. 2A and 2B. The outer tubes 62 may include a stage inlet 43 and may include one or more nozzles or openings 64 configured to allow the cooling medium to flow from the outer tubes 62 into the chamber 66, for example, such that the cooling medium is in contact with the inner tube 34. In some embodiments, one or more of the nozzles or openings 64 may be directed at an outer surface of the inner tube 34, for example, as shown in FIG. 3B. The chamber 66 may be defined between a first or upstream divider 65 and second or downstream divider 67. The upstream divider 65 may include openings that allow the cooling medium to enter the outer tubes 62. The downstream divider 67 may terminate a downstream end of each of the outer tubes 62 and may include outlets defined therein that allow the cooling medium to exit the chamber 66 and flow into another set of outer tubes 62 or exit the thermal energy recovery assembly 30. The thermal energy recovery assembly 30 may include one or more piccolo impingements 60 disposed along the length thereof. Each piccolo impingement 60, as shown in FIGS. 3A and 3B, may be considered a piccolo impingement stage with the thermal energy recovery assembly 30 including one or more piccolo impingements in series or parallel with one another.

In some embodiments, one or more of the nozzles or openings 64 may have a circular cross-section. In some such embodiments, the diameter of the one or more nozzles or openings 64 may range from about 1 millimeter (mm) to about 15 mm, for example, from about 2 mm to about 10 mm, from about 3 mm to about 8 mm, or from about 4 mm to about 7 mm. In nozzles or openings 64 not having a circular cross-section, the area of the cross-section of the nozzles or openings 64 may substantially correspond to the area of nozzles or openings 64 having a circular cross-section. In some embodiments, the nozzles or openings 64 may be circumferentially aligned relative to the respective outer tubes 62, such that fluid passing through each of the nozzles or openings 64 is directed at the outer surface of the inner tube 34, for example, at an angle of about ninety degrees relative to the outer surface of the inner tube 34. In some embodiments, one or more of the nozzles or openings 64 may be circumferentially oriented relative to its respective outer tube 62, such that fluid passing through the nozzle or opening 64 is at a non-perpendicular angle relative to the outer surface of the inner tube 34, ranging, for example, from about ten degrees to about eighty degrees, twenty degrees to about eighty degrees, thirty degrees to about eighty degrees, or about forty-five degrees to about eighty degrees. In some embodiments, the nozzles or openings 64 may be located at different points and circumferentially aligned along the length (e.g., in a direction along the longitudinal axis) of the outer tube 62. In some embodiments, nozzles or openings 64 may be spaced from the outer surface of the inner tube 34 by a distance ranging, for example, when the nozzles or openings 64 have a circular cross-section, from about a distance equal to the diameter to the nozzle or opening 64 to about twelve times the diameter of the nozzle or opening 64, or from about the diameter to about ten times the diameter, from about two times the diameter to about eight times the diameter.

In some embodiments, each of the outer tubes 62 may include a single nozzle or opening 64 at each of a plurality of locations along the length of the outer tubes 62. In some embodiments, each of the outer tubes 62 may include a number of nozzles or openings 64 ranging from one to fifteen nozzles or openings 64, from one to ten nozzles or openings 64, from one to five nozzles or openings 64 (e.g., four nozzles or openings 64) or from five to ten nozzles or openings 64. In some embodiments, the distance between nozzles or openings 64, for example, on a respective outer tube 62 may be defined, such that the distance between adjacent nozzles or openings 64 divided by diameter of the nozzle or opening 64 is greater than or equal to one and less than or equal to twenty. The number of outer tubes 62 in a stage may be between one and twelve, or between two and six.

The thermal energy recovery assembly 30 may have a modular design with a plurality of stages along a length thereof. For example, the thermal energy recovery assembly 30 may include one or more plate impingement stages 50 and one or more piccolo impingement stages 60. In some embodiments, the thermal energy recovery assembly 30 may include only plate impingement stages 50 or may include only piccolo impingement stages 60. In particular embodiments, the thermal energy recovery assembly 30 may include plate impingement stages 50, piccolo impingement stages 60, and turbulence-promoting (TP) features or TP stages.

The thermal energy recovery assembly 30 may be used in conjunction with a conventional gas to liquid steam-raising TLE. For example, a conventional TLE may be used to perform the initial quenching of the reactor effluent and then be followed by the thermal energy recovery assembly 30, as long as the reactor effluent enters the thermal energy recovery assembly 30 at a temperature of at least 550° C., at least 575° C., at least 600° C., at least 610° C., at least 620° C., at least 630° C., at least 640° C., at least 650° C., at least 700° C., at least 750° C., or at least 800° C., or at least 850° C. Alternatively, the thermal energy recovery assembly 30 may be followed by a conventional TLE, for example, if the thermal energy recovery assembly 30 preheats the feed to at least 350° C., at least 375° C., at least 400° C., at least 425° C., at least 450° C., at least 475° C., at least 500° C., at least 525° C., at least 550° C., at least 575° C., at least 600° C., at least 625° C., or at least 650° C. For example, the furnace assembly 10 may include a conventional TLE before or after the thermal energy recovery assembly 30. For example, the reactor effluent line 28 and/or the quenched effluent line 38 may include a conventional TLE. In some embodiments, the conventional gas-to-liquid steam-raising TLE may be part of the same assembly as the thermal energy recovery assembly 30. In some embodiments, the thermal energy recovery assembly 30 according to some embodiments may be combined with superheating a steam stream.

The characteristics of the stages of the thermal energy recovery assembly 30 may be tuned depending, for example, on the position of the stage within the thermal energy recovery assembly 30. For example, when the thermal energy recovery assembly 30 includes plate impingement stages 50, the channels 52 and/or 56, the nozzles or openings 54, and/or the length of the plate impingement stages 50 may be sized and dimensioned to optimize heat transfer for conditions at that position along the thermal energy recovery assembly 30. As such, the first channel 56 of a stage 50 at a first position along the thermal energy recovery assembly 30 may have a radial height greater than a radial height of a first channel 56 of a stage 50 at a second position along the thermal energy recovery assembly 30. Similarly, the nozzles or openings 54 of a stage 50 at the first position may have a diameter less than a diameter of nozzles or openings 54 of a stage 50 at a second position. In some embodiments, the radial height may remain substantially equal between one or more stages. In some embodiments, when the thermal energy recovery assembly 30 includes piccolo impingement stages 60, the diameter of the outer tubes 62, the size and/or number of the nozzles 64, and/or the length of the piccolo impingement stages 60 may be sized and dimensioned to optimize heat transfer for conditions at that position along the thermal energy recovery assembly 30. In some embodiments, the number of nozzles or openings 54 from stage to stage may be higher, lower, or the same. In some embodiments, the number of rows of nozzles or openings 54 may vary from stage to stage. The conditions along the thermal energy recovery assembly 30 may include the temperature of the reactor effluent, the temperature of the feed, the inlet and/or outlet pressure of the reactor effluent, the inlet and/or outlet pressure of the feed, the pressure drop of the reactor effluent along the length of the assembly pressure, the pressure drop of the feed along the length of the thermal energy recovery assembly 30, the temperature difference between the feed and the reactor effluent, the velocity of the reactor effluent, and/or the velocity of the feed.

As noted above, the thermal energy recovery assembly 30 may include sections that are in series with one another. The thermal energy recovery assembly 30 may activate or deactivate one or more of these sections based, for example, on the temperature of hot reactor effluent entering the thermal energy recovery assembly 30, the temperature of quenched reactor effluent exiting the thermal energy recovery assembly 30, the temperature of feed entering thermal energy recovery assembly 30, and/or the temperature of feed exiting thermal energy recovery assembly 30. In some embodiments, when a section is activated, feed may flow through the activated section, and when a section is deactivated, feed is prevented from flowing through the deactivated section. For example, when the quenched reactor effluent exiting the thermal energy recovery assembly 30 is above a desired temperature, the thermal energy recovery assembly 30 may activate one or more additional sections so that the temperature of the quenched reactor effluent exiting the thermal energy recovery assembly 30 decreases toward the desired temperature, and/or when the quenched reactor effluent exiting the thermal energy recovery assembly 30 is below a desired temperature, the thermal energy recovery assembly 30 may deactivate one or more sections so that the temperature of the quenched reactor effluent exiting the thermal energy recovery assembly 30 increases toward the desired temperature. In some embodiments, the thermal energy recovery assembly 30 may include one or more controllers configured to control operation of one or more of the sections, for example, as will be understood by those skilled in the art. For example, the thermal energy recovery assembly 30 may include a plurality of temperature sensors, pressure sensors, flow rate sensors, etc., in communication with the controller, and the controller may use control logic in the form of computer software and/or hardware programs to make control decisions associated with controlling operation of the thermal energy recovery assembly 30, for example, including the one or more sections. In some embodiments, the thermal energy recovery assembly 30 may include valves associated with the lines and/or conduits, and the controller may communicate control signals based at least in part on the control decisions to actuators associated with the valves to control the flow of fluid (e.g., gases and/or liquids) and/or heat, and the actuators may be operated according to the communicated control signals to operate the parts of the thermal energy recovery assembly 30. In some examples, the controller may be supplemented or replaced by human operators at least partially manually controlling the thermal energy recovery assembly 30 to meet desired performance parameters based at least in part on efficiency considerations.

In some embodiments, the thermal energy recovery assembly 30 may be configured to quench the hot reactor effluent within a residence time and/or with a pressure drop consistent with other quenching devices of a gas-fired cracking furnace. In some embodiments, the thermal energy recovery assembly 30 may be tuned or optimized to be substantially equivalent to, or improved relative to, other types of quenching devices. For example, the thermal energy recovery assembly 30 may be configured such that a residence time, as measured by the time within the thermal energy recovery assembly 30, is less than 100 milliseconds (ms), for example, less than 90 ms, or less than 85 ms (e.g., less than 83 ms); that a pressure drop of the reactor effluent is less than 0.35 bar, for example, less than 0.30 bar, less than 0.25 bar, or less than 0.20 bar (e.g., less than 0.15 bar); and/or that a cooling rate is greater than 2.5 degrees Kelvin (K)/ms, for example, greater than 3.5 K/ms, greater than 4.0 K/ms, greater than 4.5 K/ms, at least 5 degrees K/ms, or at least 5.5 degrees K/ms, for example, where the cooling rate may be defined as the inlet temperature of the hot reactor effluent (in degrees K) minus 923 K, divided by the residence time required for cooling from the temperature of the hot reactor effluent to 923 K, unless the inlet temperature of the hot reactor effluent (in degrees K) is less than 923 K or the temperature of the cooled reactor effluent is greater than 923K, in which case the cooling rate may be defined as the inlet temperature of the hot reactor effluent (in degrees K) minus the temperature of the cooled reactor effluent when it leaves the thermal energy recovery assembly, divided by the residence time of the effluent in the assembly. Thermal energy recovery assembly 30 may be configured to achieve, in addition to the effluent-side pressure drop and cooling rate performance, a pressure drop of the feed from 2 to 15 bar, for example, from 2.5 to 10 bar, from 3 to 8 bar, from 3 to 10 bar, or from 4 to 9 bar (e.g., from 5 to 8 bar), for example, to manage the amount of pressurization required before the feed enters the thermal energy recovery assembly 30 while promoting a sufficiently high rate of heat transfer from the inner tube to the feed.

In some embodiments, the thermal energy recovery assembly 30 may be configured and/or controlled to quench the hot reactor effluent and to preheat the hydrocarbon feed to a reactor feed temperature. For example, the thermal energy recovery assembly 30 may be configured with one or more stages to transfer heat from the hot reactor effluent to the hydrocarbon feed, which may not be preheated, or at least not preheated sufficiently to supply to a cracking furnace for cracking. In some embodiments, the stages may have a tube-in-tube design with the hot reactor effluent flowing through an inner tube and the hydrocarbon feed flowing through an outer tube. The outer tube may include stages having one or more heat transfer enhancements, such as, for example, plate impingement, piccolo impingement, one or more turbulence promoting features associated with the outer tube and/or the inner tube, and/or increased surface area associated with the outer tube and/or the inner tube. For example, the inner tube may include one or more heat transfer enhancements configured to promote heat transfer from the hot reactor effluent. Configuring the one or more stages and inner tube may include selecting stages to achieve desired properties of the hot reactor effluent while transferring heat to the cold feed. For example, stages may be selected to improve or maximize a cooling rate of the hot reactor effluent, to improve or minimize a pressure drop of the hot reactor effluent, to improve or minimize a residence time of the hot reactor effluent, and/or to improve or minimize a pressure drop of the hydrocarbon feed.

In some embodiments, the thermal energy recovery assembly may include a plurality of inner tubes parallel with one another, with each inner tube disposed within an outer tube and each outer tube having one or more heat transfer enhancements to enhance heat transfer from the inner tube to an annulus defined within the outer tube. In some embodiments, the thermal energy recovery assembly may include a plurality of inner tubes parallel with one another, with each inner tube disposed within an outer tube, and with the outer tube and optionally the inner tube having one or more heat transfer enhancements to enhance heat transfer from the inner tube to an annulus defined within the outer tube.

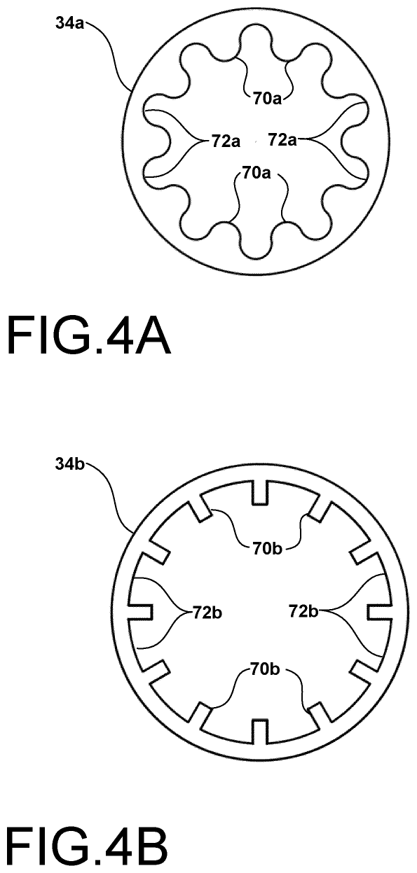

FIG. 4A is a schematic section view of an example inner tube 34a including example rounded projections 70a on an interior surface 72a of the inner tube 34a, according to embodiments of the disclosure. As shown in FIG. 4A, in some embodiments, the interior surface 72a of the inner tubes 34 may include turbulence-promoting structures and/or structures to increase the surface area of the interior surface 72a. For example, as shown in FIG. 4A, the interior surface 72a of the inner tube 34a may include one or more rounded projections 70a. In some embodiments, the one or more rounded projections 70a may extend toward the center of the inner tube 34a and/or may extend longitudinally, partially, intermittently, or fully, along the length of the inner tube 34a. In some embodiments, the rounded projections 70a may be the same or differ from one another. In some embodiments, the one or more rounded projections 70a may extend helically along the longitudinal length of the inner tube 34a, for example, to promote swirling of the flow through the inner tube 34a. In some embodiments, projections on the interior surface of inner tubes 34 may have a non-rounded configuration. For example, FIG. 4B is a schematic section view of another example inner tube 34b including example rectangular projections 70b on an interior surface 72b of the inner tube 34b, according to embodiments of the disclosure. In some embodiments, the interior surface of the inner tubes 34 may include a combination of rounded projections and rectangular projections. Other configurations of projections are contemplated. In some embodiments, the inner tube 34 may include turbulence promoting structures and/or structures to increase the surface area of an outer surface of the inner tube 34. For example, turbulence promoting structures and/or structures to increase the surface area of the outer surface of the inner tube 34 may include projections at least similar to the above-noted projections on the interior surface of the inner tube 34. In some embodiments, structures to increase the surface area of the outer surface of the inner tube 34 may be configured to enhance the effectiveness of the turbulence-promoting structures and/or impingement features. In some embodiments, the inner surface of the outer tube 40 may include surface area enhancements, for example, such as those described above.

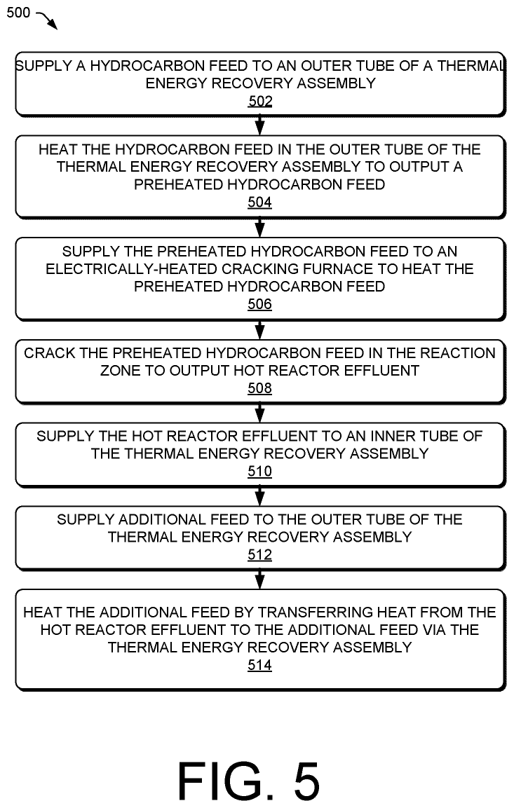

FIG. 5 is a block diagram of an example method 500 to heat a hydrocarbon feed including, for example, one or more of ethane, propane, butane, condensate, light naphtha, heavy naphtha, gas oil, pyrolysis oil, materials derived from processing refinery streams, Fischer-Tropsch products, plastic waste, and/or biofeedstocks. The hydrocarbon feed may additionally include steam. The hydrocarbon feed may be preheated and thereafter cracked in an electrically heated cracking furnace, which may output cracked hydrocarbons including olefins. The example method 500, according to some embodiments, is illustrated in FIG. 5 as a collection of blocks in a logical flow graph, which represent a sequence of operations. The order in which the operations are described is not intended to be construed as a limitation, and any number of the described blocks may be combined in any order and/or in parallel to implement the method. Moreover, the operations described in one or more of the blocks may be optional and/or omitted from the example method 500, such as, for example, the operations described by blocks 512 and/or 514, although one or more of the operations described by other blocks may also, or alternatively, be omitted from the example method 500.

The example method 500, at 502, may include supplying a hydrocarbon feed to an outer tube of a thermal energy recovery assembly. For example, the thermal energy recovery system may include any thermal energy recovery systems described herein. As noted above, the hydrocarbon feed may include, for example, one or more of ethane, propane, butane, condensate, light naphtha, heavy naphtha, gas oil, pyrolysis oil, and/or materials derived from processing refinery streams, Fischer-Tropsch products, plastic waste, or biofeedstocks, or any other hydrocarbons that may be converted to olefins in a cracking process, and may additionally include steam. The hydrocarbon feed may, in some embodiments, include, or be, a hydrocarbon feed supplied by a source of hydrocarbon feed.

At 504, the example method 500 further may include heating the hydrocarbon feed in the outer tube of the thermal energy recovery assembly to output a preheated hydrocarbon feed. For example, as explained herein, the hydrocarbon feed may be preheated via heat transfer in the thermal energy recovery system with the thermal energy being at least partially supplied by hot reactor effluent of the cracking process.

The example method 500, at 506 also may include supplying the preheated hydrocarbon feed to an electrically heated cracking furnace including a reaction zone to heat the preheated hydrocarbon feed, for example, as previously described herein.

At 508, the example method 500 further may include cracking the preheated hydrocarbon feed in the reaction zone to output hot reactor effluent including cracked hydrocarbons and olefins, for example, as described previously herein.

The example method 500, at 510, also may include supplying the hot reactor effluent to an inner tube of the thermal energy recovery assembly, for example, as previously described herein. For example, in some embodiments, supplying the hot reactor effluent to the inner tube of the thermal energy recovery assembly may include supplying the hot reactor effluent to the inner tube of the thermal energy recovery assembly at a temperature of at least 3500 Celsius, at least 3750 Celsius, at least 400° Celsius, at least 4250 Celsius, at least 4500 Celsius, at least 4750 Celsius, at least 5000 Celsius, at least 5250 Celsius, at least 5500 Celsius, at least 5750 Celsius, at least 6000 Celsius, at least 6250 Celsius, at least 6500 Celsius, at least 700° C., at least 750° C., at least 800° C., or at least 850° C.

At 512, the example method 500 further may include supplying additional feed to the outer tube of the thermal energy recovery assembly. The additional feed may, in some embodiments, be a continuation of the supply of hydrocarbon feed at 502 from the hydrocarbon feed source, a different hydrocarbon feed, water, or steam. The additional feed may be supplied to a section different from the section to which the hydrocarbon feed is supplied. The additional feed may mix with the hydrocarbon feed, the mixed feed exiting the thermal energy recovery assembly at a common outlet. The additional feed may traverse different stages than does the hydrocarbon feed, and exit through a different outlet.

The example method 500, at 514, also may include heating the additional feed by transferring heat from the hot reactor effluent to the additional hydrocarbon feed via the thermal energy recovery assembly, for example, as described previously herein. Heating the additional feed in the outer tube of the thermal energy recovery assembly to output a preheated feed may include heating the feed to a temperature of at least 3500 Celsius, at least 3750 Celsius, at least 4000 Celsius, at least 4250 Celsius, at least 4500 Celsius, at least 4750 Celsius, at least 5000 Celsius, at least 5250 Celsius, at least 5500 Celsius, at least 5750 Celsius, at least 6000 Celsius, at least 6250 Celsius, or at least 6500 Celsius. In some embodiments, supplying the hot reactor effluent to the inner tube of the thermal energy recovery assembly may include quenching the hot reactor effluent via heat transfer to the additional hydrocarbon feed, for example, as described herein. In some embodiments, heating the feed in the outer tube of the thermal energy recovery assembly may include preheating the feed via heat transfer to the feed from the hot reactor effluent. In some embodiments, the example method 500 may further include enhancing the heat transfer to the additional feed by providing heat transfer enhancements on one or more of the outer tube or the inner tube. The heat transfer enhancements may include one or more of plate impingement, piccolo impingement, turbulence promotion, or increased surface area, for example, as previously described herein.

In an embodiment of the present disclosure, a method to produce olefins may include one or more cooling steps characterized by: supplying a hydrocarbon feed to an outer tube of a thermal energy recovery assembly; heating the hydrocarbon feed in the outer tube of the thermal energy recovery assembly to output a preheated hydrocarbon feed; supplying the preheated hydrocarbon feed to an electrically powered cracking furnace comprising a reaction zone to heat the preheated hydrocarbon feed; cracking the preheated hydrocarbon feed in the reaction zone of the electrically heated cracking furnace using heat generated by electricity to output hot reactor effluent comprising cracked hydrocarbons and olefins; supplying the hot reactor effluent to an inner tube of the thermal energy recovery assembly; and cooling the hot reactor effluent in the inner tube of the thermal energy recovery assembly by transferring heat to the hydrocarbon feed; configuring the thermal energy recovery such to operate within preferred operating regimes without performance loss through feed pre-cracking and/or effluent coking.

In certain embodiments, the thermal heat recovery assembly may include one or more sections in series or parallel configuration, with each of them having either counter or co-current flow direction. The detailed layout of these sections, and of the stages contained within these sections, in terms of the tube and annulus layout, the heat transfer enhancement methods, the heat transfer area enhancement methods may differ in parts or entirely.

In certain embodiments of the thermal energy recovery assembly, the individual stages or sections may be configured and sized such that the thermal energy recovery operates within preferred operating regimes without significant or any performance losses. The latter may occur, when through chemical and/or physical processes fouling layers are generated on the inner tube inside or inner tube outside surfaces reducing the heat transfer from the inner tube to the annulus. Beside the type and composition of the hydrocarbon feed and the composition of the hot reactor effluent, these chemical and physical processes heavily depend on the temperature prevailing on the inner tube inside and respective outside surface. Such kinds of processes may be feed pre-cracking in the annulus and chemically and physically induced coking on the effluent side. Feed pre-cracking in the annulus defines an upper temperature level, which should not be exceeded on the inner tube outside surface. An additional upper temperature level, which should not be exceeded on the inner tube inside surface may be defined by accelerated chemical coking at high surface temperature. A lower temperature level, which should not be reached on the inner tube inside surface may be defined by coking driven by the condensation of heavy components in the effluent. The upper temperature level defined by the risk of pre-cracking initiation in the annulus may be dependent on the hydrocarbon feedstock type. The upper temperature level may be, for example, 530° C., 540° C., 550° C., 600° C., 640° C., 650° C., 660° C., 680° C., 700° C., 720° C., 750° C., etc. For ethane type feedstock this upper temperature level may be 700-720° C. For propane type feedstock this upper temperature level may be 680-700° C. For butane type feedstock this upper temperature level may be 660-680° C. For naphtha type feedstock this upper temperature level may be 640-660° C. For feedstocks heavier than naphtha this upper temperature level may be 540-640° C. The upper temperature level defined by the risk of enhanced chemical coking at the inner tube inside surface may be determined by the effluent reactivity at temperatures above 650-680° C. The lower temperature limit defined by enhanced coking due to the condensation of heavy components of the effluents may be determined by the feed type and the cracking conditions. The lower temperature level may be, for example, 150° C., 160° C., 170° C., 180° C., 200° C., 250° C., 270° C., 300° C., 350° C., etc. For ethane type feedstock the lower temperature level may be 160-220° C. For propane type feedstock the lower temperature level may be 170-250° C. For butane type feedstock the lower temperature level may be 180-270° C. For naphtha type feedstock the lower temperature level may be 270-320° C. For feedstocks heavier than naphtha this lower temperature level may be 270-320° C.

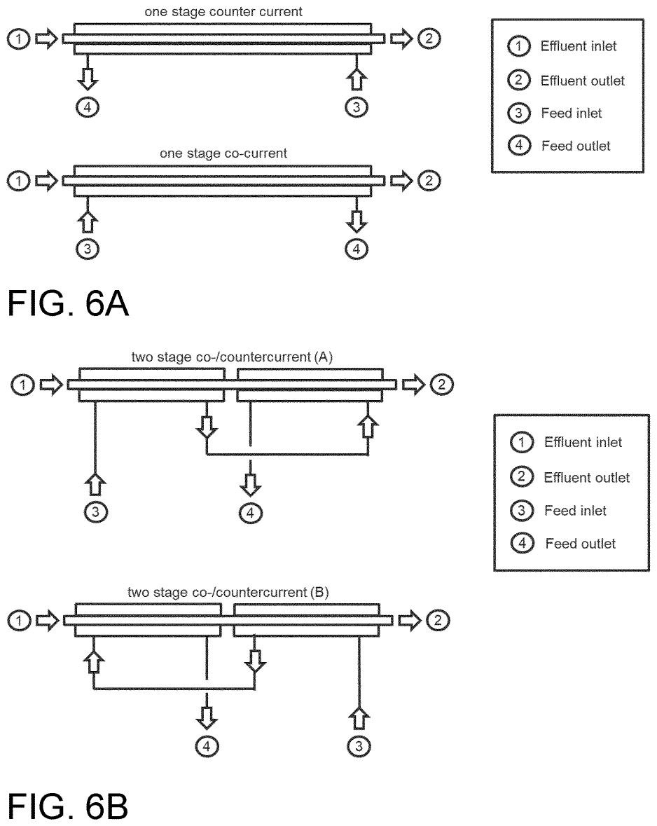

With reference to FIG. 6A for a single-section application of the tube-in-tube gas-gas exchanger within the energy recovery assembly, the reactor effluent may be either in countercurrent or co-current flow direction to the feed. The hot reactor effluent may be supplied to the inner tube and the feed to the outer tube disposed about the inner tube to enclose an annulus. The annulus may include at least one heat transfer enhancement to enhance heat transfer from the inner tube to the annulus through impingement, turbulence promotion, high shear inducing geometry, or increased surface area.

With reference to FIG. 6B for a two-section application of the tube-in-tube gas-gas exchanger within the energy recovery assembly, the flow direction of the reactor effluent may be co-current with the feed in a first section and countercurrent to the feed in a second section. The hot reactor effluent may be supplied to the inner tube and the feed to the outer tube disposed about the inner tube to enclose an annulus. The annulus may include at least one heat transfer enhancement to enhance heat transfer from the inner tube to the annulus through impingement, turbulence promotion, high shear inducing geometry, or increased surface area. The heat transfer enhancement method(s) applied and the detailed layout may be identical for both sections or may be different.

In some embodiments of the two-section application, the cold feed may enter the tube-in-tube gas-gas exchanger at the upstream co-current section, in which the first preheating takes place, and may be further heated in the downstream countercurrent section. The hot reactor effluent may be supplied to the inner tube and the feed to the outer tube disposed about the inner tube to enclose an annulus. The annulus may include at least one heat transfer enhancement to enhance heat transfer from the inner tube to the annulus through impingement, turbulence promotion, high shear inducing geometry, or increased surface area. The heat transfer enhancement method(s) applied and the detailed layout may be identical for both sections or may be different.

In some embodiments of the two-section application the cold feed may enter the tube-in-tube gas-gas exchanger at the downstream countercurrent section, in which the first preheating takes place, and may be further heated in the upstream co-current section. The hot reactor effluent may be supplied to the inner tube and the feed to the outer tube disposed about the inner tube to enclose an annulus. The annulus may include at least one heat transfer enhancement to enhance heat transfer from the inner tube to the annulus through impingement, turbulence promotion, high shear inducing geometry, or increased surface area. The heat transfer enhancement method(s) applied and the detailed layout may be identical for both sections or may be different.

In some embodiments, the co-current and countercurrent sections may be implemented in a single piece of equipment. In some embodiments, the co-current and countercurrent sections may be implemented as different pieces of equipment, for example, the co-current stage may be built as a tube-in-tube exchanger with heat transfer enhancements, and the countercurrent stage may be built as a conventional tube-in-tube or shell-and-tube type heat exchanger, or any other suitable conventional heat exchanger design.

### Examples

The heat transfer performance of several thermal energy recovery assemblies that include heat transfer enhancements according to embodiments of this disclosure was compared to the performance of a conventional tube-in-tube gas-gas heat exchanger. The conventional heat exchanger was designed for a feed-side pressure drop of 1.76 bar, and did not include enhancements in either an inner tube or an annulus. The thermal energy recovery assemblies according to embodiments of the disclosure were as follows: (1) a tube-in-tube thermal energy recovery assembly without heat transfer enhancement in an inner tube and with high-shear geometry in an annulus, (2) a thermal energy recovery assembly including fins in the inner tube and turbulence-promoting features in the annulus, (3) a thermal energy recovery assembly including fins in the inner tube and plate impingement in the annulus, (4) a thermal energy recovery assembly including fins in the inner tube and piccolo impingement in the annulus, (5) a thermal energy recovery assembly including a plain tube (no internal fins) and turbulence promoting features in the annulus, (6) a thermal energy recovery assembly including a plain tube and plate impingement in the annulus, and (7) a thermal energy recovery assembly including a plain tube and piccolo impingement in the annulus.

For the purpose of the comparison, boundary conditions for the conventional heat exchanger (“Comp.”) and each of the seven example thermal energy recovery assemblies according to embodiments of the disclosure (1 through 7) were established as follows: hot effluent resulting from steam cracking of ethane was passed through an inner tube, and cold feed including ethane and steam was passed through an outer tube. The hot effluent mass flow rate was 351.6 kilograms/hour, and the outer diameter of the inner tube was 60.3 mm, with a tube wall thickness of 3.6 mm. The hot effluent and the cold feed were flowing according to a counter-current flow with the following inlet and outlet temperatures: T_{in, hot} equals 827 degrees C.; T_{out, hot} equals 486 degrees C.; T_{in, cold} equals 236 degrees C.; and T_{out, cold} equals 650 degrees C.

Software tools designed for calculation of heat transfer were used to assess the performance of the conventional heat exchanger (“Comp.”) and the seven examples according to embodiments of the disclosure. Table A below shows the comparative performance in terms of various metrics explained below. For each metric, the value for each of the thermal energy recovery assemblies according to embodiments of the disclosure (1 through 7) is listed relative to the corresponding value for the conventional heat exchanger. The example metrics provided for comparison are the effluent cooling rate, the heating surface area, the pressure drops on the feed and effluent sides of the corresponding device, and the effluent residence time.

TABLE A

| Device | Comp. | 1 | 2 | 3 | 4 | 5 | 6 | 7 |
|---|---|---|---|---|---|---|---|---|
| Annulus enhancement | None | High- | TP | Plate | Piccolo | TP | Plate | Piccolo |
| | | shear | | | | | | |
| Inner tube enhancement | None | None | Finned | Finned | Finned | None | None | None |
| Relative cooling rate | 1 | 1.08 | 1.47 | 1.51 | 1.58 | 1.14 | 1.16 | 1.22 |
| Relative heating surface | 1 | 0.91 | 0.74 | 0.71 | 0.70 | 0.87 | 0.85 | 0.80 |
| Relative feed pressure drop | 1 | 2.24 | 3.40 | 3.92 | 3.48 | 3.77 | 3.66 | 3.47 |
| Relative effluent pressure | 1 | 0.93 | 1.23 | 1.18 | 1.26 | 0.90 | 0.88 | 0.84 |
| drop | | | | | | | | |
| Relative residence time | 1 | 0.91 | 0.69 | 0.68 | 0.65 | 0.87 | 0.85 | 0.80 |

As shown in Table A, thermal energy recovery assemblies according to embodiments of the disclosure may provide improved performance, for example, in terms of cooling rate, residence time, effluent pressure drop, and/or required cooling surface area as compared to a heat exchanger without such heat transfer enhancement features. As will be clear from this disclosure, higher values of the cooling rate and lower values of the required surface area, the effluent pressure drop, and the effluent residence time may be generally favorable in terms of process performance and/or equipment cost.

### Examples 2(a) Through 2(e)

Heating and cooling profiles for a furnace mixed feed (i.e., steam plus naphtha) of 876.8 tons/hour (t/h) and the reaction zone effluent stream (876.8 t/h) were generated using a process simulation software tool, along with the energy requirement of the steam cracking reaction. Feasible energy balances with and without using a thermal energy recovery assembly were constructed. According to a model for a full plant, unit operations downstream of a cracker have a net heat input demand of 163 Megawatts (MW). In addition, it was determined that there is a demand for 151 MW of work for compressors and pumps that may be supplied by recovered steam using a condensing steam turbine with an efficiency of 41% or by electricity at an efficiency of 95%.

Example 2(a) is a comparative example where 120 bar steam is generated in a conventional transfer line exchanger (TLE). The mixed feed temperature is initially at 180 degrees C. because available heat recovery from the downstream of the plant can provide this starting temperature. The mixed feed may be heated from 180 degrees C. to 300 degrees C. using the 120 bar steam, since the saturated steam temperature is 324 degrees C. The remaining steam, which constitutes 244 MW of the 314 MW of heat removed from the TLE, is used to supply the 163 MW of downstream heating duty and for 33 MW of mechanical work (i.e., calculated as follows: (244 MW-163 MW) multiplied by 0.41, which equals 33 MW); the remaining 118 MW of work is supplied by 124 MW of electricity (i.e., calculated as follows: 118 divided by 0.95 equals 124 MW). In this comparative Example 2(a), there is no direct feed-effluent heat exchange. The feed is heated from 300 degrees C. to 650 degrees C. electrically, and the cracking reaction is driven by electric heating. In this comparative example, 682 MW of electricity is needed for preheating in addition to the reaction, resulting in a total electricity usage in this comparative example of 806 MW.

Example 2(b), an example according to embodiments of the disclosure, the mixed feed is preheated to 450 degrees C. by feed-effluent heat exchange using a thermal energy recovery assembly according to embodiments of the disclosure. The remaining cooling of the cracked gas is achieved by steam generation, recovering 145 MW, which may provide the energy for all but 18 MW of the downstream heating; all of the mechanical work may be performed electrically, which requires 159 MW of electricity according to this example. The feed is heated from 450 degrees C. to 650 degrees C. electrically, and the cracking reaction is driven by electric heating. The total electricity needed for the feed in addition to the furnace heating in this example is 583 MW, resulting in a total electricity usage of 760 MW.

In Example 2(c), a further example according to embodiments of the disclosure, the mixed feed may be heated to the relatively higher temperature of 550 degrees C. using the thermal energy recovery assembly according to embodiments of the disclosure. As the amount of heat transferred from the thermal energy recovery assembly to the feed is increased, the amount of steam generation decreases, and the amount of electricity required for the feed and cracking reaction decreases. In Example 2(c), the feed is heated from 550 degrees C. to 650 degrees C. electrically, and the cracking reaction is driven by electric heating. In this example, 72 MW of steam are generated, which may be used to supply some of the downstream heating; all of the mechanical work may be performed done electrically. The total electricity needed for the feed in addition to the furnace heating in this example is 511 MW, resulting in a total electricity usage of 760 MW.

In Example 2(d), another example according to embodiments of the disclosure, the feed preheat is increased to 650° C. and the steam generation is eliminated. A small adjustment to the hot side target temperature down from 400 degrees C. to 392 degrees C. marks the enthalpy balance point with this feed and target preheating condition. In Example 2(d), 433 MW of electricity is required at the furnace. No steam is generated, so 163 MW of electricity are required for the downstream heating, and 159 MW of electricity are required for the mechanical work, resulting in a total electricity consumption of 755 MW.

In Example 2(e), yet a further example according to embodiments of the disclosure, steam generation is employed only to the extent that the steam is used for mixed feed preheating to 300 degrees C.; the remainder of the preheating is achieved using a tube-in-tube exchanger according to embodiments of the disclosure. There is no steam export. The total electric heating duty at the furnace is 433 MW, which is the same as for Example 2(d)), and 250 MW less than the comparative example (i.e., Example 2(a)). In Example 2(e), 163 MW of electricity are required for the downstream heating, and 159 MW of electricity are required for the mechanical work, resulting in a total electricity consumption of 755 MW, or 51 MW less than in the comparative example (i.e., Example 2(a)).

A summary of the heat and electricity usage for Examples 2(a) through 2(e) is provided in TABLE B below. Comparing the comparative Example 2(a) with Examples 2(b) through 2(e), examples according to embodiments of the disclosure, shows that the total amount of electricity required to operate the cracking process is reduced when the feed is preheated to a temperature of at least 450° C. using example thermal energy recovery assemblies according to embodiments of the disclosure. This demonstrates that thermal energy recovery assemblies consistent with embodiments of the disclosure may promote an increased efficiency, for example, when a sufficiently high level of preheat is achieved. Among Examples 2(b) through 2(e), the electricity requirement may remain essentially the same, showing that thermal energy recovery assemblies consistent with embodiments of the disclosure may be used flexibly without loss of efficiency, for example, as long as a minimum level of preheat is accomplished.

TABLE B

| | | Example | | | | | | | | | |
|---|---|---|---|---|---|---|---|---|---|---|---|
| | | 2(a) | | 2(b) | | 2(c) | | 2(d) | | 2(e) | |
| | | T | Duty | T | Duty | T | Duty | T | Duty | T | Duty |
| | | ° C. | MW | ° C. | MW | ° C. | MW | ° C. | MW | ° C. | MW |
| Reaction Zone Effluent | ↑ | 820 | | 820 | | 820 | | 820 | | 820 | |
| Cracking | | | 433 | | 433 | | 433 | | 433 | | 433 |
| | ↑ | 650 | | 650 | | 650 | | 650 | | 650 | |
| Electric mixed feed preheat | | | 250 | | 151 | | 78 | | 0 | | 0 |
| | ↑ | 300 | | 450 | | 550 | | 650 | | 650 | |
| Heat Exchange | | | 0 | | 169 | | 242 | | 320 | | 250 |
| | ↑ | 300 | | 180 | | 180 | | 180 | | 300 | |
| Steam mixed feed preheat | | | 70 | | 0 | | 0 | | 0 | | 70 |
| Cold Feed | ↑ | 180 | | 180 | | 180 | | 180 | | 180 | |
| Reaction Zone Effluent | ↓ | 820 | | 820 | | 820 | | 820 | | 820 | |
| Heat Exchange | | | 0 | | 169 | | 242 | | 320 | | 250 |
| | ↓ | 820 | | 606 | | 506 | | 392 | | 495 | |
| Steam Generation | | | 314 | | 145 | | 72 | | 0 | | 70 |
| Quenched Cracked Gas | ↓ | 400 | | 400 | | 400 | | 392 | | 392 | |
| Steam available for downstream heat and work | | | 244 | | 145 | | 72 | | 0 | | 0 |
| Electric Heating (preheat + reaction) | | | 682 | | 583 | | 511 | | 433 | | 433 |
| Electricity needed for downstream heating | | | 0 | | 18 | | 91 | | 163 | | 163 |
| Electricity required for downstream work | | | 124 | | 159 | | 159 | | 159 | | 159 |
| Total Electricity required | | | 806 | | 760 | | 760 | | 755 | | 755 |

### Examples 3(a) Through 3(e)

Examples 3(a) through 3(e) are analogous to Examples 2(a) through 2(e) above, except that the order of heat exchange is reversed on the hot cracked gas cooling stream. Steam generation is used for the first part of the cooling and feed-effluent heat exchange is used for the secondary cooling. A summary of the results of Examples 3(a) through 3(e) is given in TABLE C below. As with Examples 2(a) through 2(e), the results show the total electricity consumed by the cracking process may be decreased through the use of thermal energy recovery assemblies consistent with embodiments of the disclosure. In addition, comparison of the results in Tables B and C shows that under a wide range of conditions, an equivalent electricity consumption may be achieved using different sequences combining steam raising and use of thermal energy recovery assemblies consistent with embodiments of the disclosure.

TABLE C

| | | Example | | | | | | | | | |
|---|---|---|---|---|---|---|---|---|---|---|---|
| | | 3(a) | | 3(b) | | 3(c) | | 3(d) | | 3(e) | |
| | | T | Duty | T | Duty | T | Duty | T | Duty | T | Duty |
| | | ° C. | MW | ° C. | MW | ° C. | MW | ° C. | MW | ° C. | MW |
| Reaction Zone Effluent | ↑ | 820 | | 820 | | 820 | | 820 | | 820 | |
| Cracking | | | 433 | | 433 | | 433 | | 433 | | 433 |
| | ↑ | 650 | | 650 | | 650 | | 650 | | 650 | |
| Electric mixed feed preheat | | | 250 | | 151 | | 78 | | 0 | | 0 |
| | ↑ | 300 | | 450 | | 550 | | 650 | | 650 | |
| Heat Exchange | | | 0 | | 169 | | 242 | | 320 | | 250 |
| | ↑ | 300 | | 180 | | 180 | | 180 | | 300 | |
| Steam mixed feed preheat | | | 70 | | 0 | | 0 | | 0 | | 70 |
| Cold Feed | ↑ | 180 | | 180 | | 180 | | 180 | | 180 | |
| Reaction Zone Effluent | ↓ | 820 | | 820 | | 820 | | 820 | | 820 | |
| Steam Generation | | | 314 | | 145 | | 72 | | 0 | | 70 |
| | ↓ | 400 | | 638 | | 731 | | 820 | | 734 | |
| Heat Exchange | | | 0 | | 169 | | 242 | | 320 | | 250 |
| Quenched Cracked Gas | ↓ | 400 | | 400 | | 400 | | 392 | | 392 | |
| Steam available for downstream heat and work | | | 244 | | 145 | | 72 | | 0 | | 0 |
| Electric Heating (preheat + reaction) | | | 682 | | 583 | | 511 | | 433 | | 433 |
| Electricity needed for downstream heating | | | 0 | | 18 | | 91 | | 163 | | 163 |
| Electricity required for downstream work | | | 124 | | 159 | | 159 | | 159 | | 159 |
| Total Electricity required | | | 806 | | 760 | | 760 | | 755 | | 755 |

### Examples 4(a)-4(c)

In examples 4(a) through 4(c), for an ethane application case, a feed-effluent heat exchanger is used for the primary cooling and steam generation is used for the secondary cooling. The examples 4(a) to 4(c) differ with respect to the configuration of the feed-effluent heat exchanger. In example 4(a) the feed-effluent heat exchanger comprises one section and is countercurrent (i.e., is fully countercurrent). In examples 4(b) and 4(c) the feed-effluent heat exchanger comprises two sections with a first co-current and a second countercurrent section. In example 4(b) the cold feed is routed to the co-current section first and then routed to the counter-current section. In example 4(c) the cold feed is routed to the counter-current section first and then routed to the co-current section. In all three examples 4(a) through 4(c) the reactor effluent flow and the feed flow are identical. In all three examples 4(a) to (c) the feed is heated from 230° C. to 580° C. and the reactor effluent cooled from 849° C. to 561° C. The configurations of examples 4(a) to(c) were not subjected to economic optimization, and are presented here primarily to illustrate the possible trade-offs between different conceptual configurations. A summary of the results of examples 4(a) through 4(c) is given in TABLE D below. Tube metal temperatures for each metric are given in ° C., while the values of other metrics are listed relative to the corresponding values of example 4(a). These comparative metrics are the heat transferred, the inside tube pressure drop (i.e., the effluent pressure drop), the effluent residence time, the cooling rate and the required heating surface area.

The highest maximum tube surface temperatures at the inner tube are expected for example 4(a), while the lowest maximum tube surface temperatures are expected for example 4(b). As a consequence, the risk to enter regimes where coking may occur due to pre-cracking at the tube outside surface and enhanced chemical coking at the tube inside surface is highest for the configuration represented by example 4(a). The resulting tube surface temperatures of example 4(c) are in between example 4(a) and 4(b), but closer to 4(b). At the cold end of the assembly the lowest tube inside temperatures are expected for example 4(a) and 4(c), while example 4(b) exhibits higher tube inside temperatures. For the ethane application examples shown herein the risk of significant condensation coking is low for any of examples 4(a)-4(c).

With respect to the tube inside effluent pressure drop, which has impact on the cracking selectivity, it is lowest for example 4(a), followed by example 4(c) and is significantly higher for example 4(b). The high effluent pressure drop in example 4(b) results from the significantly higher tube length compared to example 4(a) and 4(c).

With respect to residence time and cooling rate, both referring to cooling of the effluent until it reaches a temperature of 650° C., the most favorable values are seen for the configuration of example 4(b), which has the highest temperature difference between effluent and feed at the effluent inlet.

Taking into account the high required heat transfer surface of example 4(b) and the high tube metal temperatures seen for example 4(a), the configuration as defined for example 4(c) may be the best solution for the application case shown herein.

TABLE D

| Example | | 4(a) | 4(b) | 4(c) |
|---|---|---|---|---|
| Heat transferred | [%] | 100 | 100 | 100 |
| Tube inside pressure drop | [%] | 100 | 176 | 108 |
| Tube metal temperatures | | | | |
| max. inside tube | [° C.] | 668 | 600 | 615 |
| max. outside tube | [° C.] | 644 | 595 | 605 |
| min. inside tube | [° C.] | 331 | 451 | 333 |
| residence time | [%] | 100 | 87 | 112 |
| cooling rate | [%] | 100 | 115 | 89 |
| Heat transfer surface | [%] | 100 | 193 | 108 |

### Examples 5(a) Through 5(c)

In examples 5(a) through 5(c), for a naphtha application case, a feed-effluent heat exchanger is used for the primary cooling, and steam generation is used for the secondary cooling. The examples 5(a) to 5(c) differ with respect to the configuration of the feed-effluent heat exchanger. In example 5(a) the feed-effluent heat exchanger comprises one section and is countercurrent (i.e., is fully countercurrent). In examples 5(b) and 5(c) the feed-effluent heat exchanger comprises two sections with a first co-current and a second countercurrent section. In example 5(b) the cold feed is routed to the co-current section first and then routed to the counter-current section. In example 5(c) the cold feed is routed to the counter-current section first and then routed to the co-current section. In all three examples 5(a) through 5(c) the reactor effluent flow and the feed flow are identical. In all three examples 5(a) to (c) the feed is heated from 223° C. to 594° C. and the reactor effluent cooled from 862° C. to 539° C. The configurations of examples 5(a) to (c) were not subjected to economic optimization, and are presented here primarily to illustrate the possible trade-offs between different conceptual configurations. A summary of the results of examples 5(a) through 5(c) is given in TABLE E below. The tube metal temperatures for each metric are given in ° C., while the value for other metrics are listed relative to the corresponding values of example 5(a). These comparative metrics are the heat transferred, the inside tube pressure drop (i.e., effluent pressure drop), the effluent residence time, the cooling rate and the required heating surface area.

The highest maximum tube surface temperatures at the inner tube are expected for example 5(a), while the lowest maximum tube surface temperatures are expected for example 5(b). As a consequence the risk to enter regimes where coking may occur due to pre-cracking at the tube outside surface and enhanced chemical coking at the tube inside surface is highest for the configuration represented by example 5(a). The resulting tube surface temperatures of example 5(c) are in between example 5(a) and 5(b), but closer to 5(b). At the cold end of the assembly the lowest tube inside temperatures are expected for example 5(a) and 5(c), while example 5(b) exhibits higher tube inside temperatures. For the examples 5(a) and 5(c) the distance to the temperature below which risk of enhanced condensation coking increases is lowest.

With respect to the effluent pressure drop, which has an impact on the cracking selectivity, it is lowest for example 5(a), followed by example 5(c) and is significantly higher for example 5(b). The high effluent pressure drop in example 5(b) results from the significantly higher tube length compared to example 5(a) and 5(c).

TABLE E

| Example | | 5(a) | 5(b) | 5(c) |
|---|---|---|---|---|
| Heat transferred | [%] | 100 | 100 | 100 |
| Tube inside pressure drop | [%] | 100 | 217 | 111 |
| Tube metal temperatures | | | | |
| max. inside tube | [° C.] | 674 | 609 | 617 |
| max. outside tube | [° C.] | 654 | 605 | 613 |
| min. inside tube | [° C.] | 309 | 435 | 310 |
| residence time | [%] | 100 | 83 | 120 |
| cooling rate | [%] | 100 | 120 | 79 |
| Heat transfer surface | [%] | 100 | 243 | 112 |

With respect to residence time and cooling rate, both referring to cooling of the effluent until a temperature of 650° C. is reached, the most favorable values are seen for the configuration of example 5(b), which has the highest temperature difference between effluent and feed at the effluent inlet.

Taking into account high required heat transfer surface of example 5(b) and the high tube metal temperatures seen for example 5(a), the configuration as defined for example 5(c) may be the best solution for the application case shown herein, as long as a minimum inside tube temperature of 310° C. is acceptable for this naphtha feedstock. If a higher temperature is required to prevent condensation coking, then the configuration as defined for example 5(b) may be the preferred solution. Generally, the skilled person will select from the configurations of examples 5(a) through 5(c) depending on specific requirements, technical and economic boundary conditions.

Having now described some illustrative embodiments of the disclosure, it should be apparent to those skilled in the art that the foregoing is merely illustrative and not limiting, having been presented by way of example only. Numerous modifications and other embodiments are within the scope of one of ordinary skill in the art and are contemplated as falling within the scope of the disclosure. In particular, although many of the examples presented herein involve specific combinations of method acts or system elements, it should be understood that those acts and those elements may be combined in other ways to accomplish the same objectives. Those skilled in the art should appreciate that the parameters and configurations described herein are exemplary and that actual parameters and/or configurations will depend on the specific application in which the systems and techniques of the disclosure are used. Those skilled in the art should also recognize or be able to ascertain, using no more than routine experimentation, equivalents to the specific embodiments of the disclosure. It is, therefore, to be understood that the embodiments described herein are presented by way of example only and that, within the scope of any appended claims and equivalents thereto, the embodiments of the disclosure may be practiced other than as specifically described.

Furthermore, the scope of the present disclosure shall be construed to cover various modifications, combinations, additions, alterations, etc., above and to the above-described embodiments, which shall be considered to be within the scope of this disclosure. Accordingly, various features and characteristics as discussed herein may be selectively interchanged and applied to other illustrated and non-illustrated embodiment, and numerous variations, modifications, and additions further can be made thereto without departing from the spirit and scope of the present disclosure as set forth in the appended claims.

An example thermal energy recovery assembly A to recover thermal energy from hot reactor effluent to heat a feed to an electrically powered reactor furnace, may include an inner tube having a first inlet configured to receive hot reactor effluent from an electrically powered reactor furnace, and an outer tube disposed about the inner tube to enclose an annulus about the inner tube. The annulus may have a second inlet configured to receive a feed to the electrically powered reactor furnace, and the annulus may be configured to use the feed for the electrically powered reactor furnace as a cooling medium to recover thermal energy from the hot reactor effluent prior to the feed being supplied to the electrically powered reactor furnace. The annulus may be configured to enhance heat transfer from the hot reactor effluent to the feed in the annulus.

In some embodiments, hot reactor effluent may reach a first inlet via a gas inlet chamber or other connector. In some embodiments, cooling may be supplied to the gas inlet chamber or other connector. In some embodiments, the gas chamber may connect one or more than one cracking coil to one or more than one inner tube. In some embodiments, a header may be provided to connect the feed to more than one annulus. In some embodiments, cooled cracked gas from more than one inner tube may be collected using a header. In some embodiments, heated feed from more than one annulus may be combined via a header. In some embodiments, multiple annuli may be contained in a single mechanical device, which may receive hot effluent from multiple cracking coils via a gas inlet chamber or other connector, and cold feed from a feed header.

The example assembly A above, wherein the outer tube includes at least one heat transfer enhancement to enhance heat transfer from the inner tube to the annulus.

The example assembly A above, wherein the outer tube includes a first stage, and the at least one heat transfer enhancement comprises one or more of a plate impingement, a piccolo impingement, turbulence promotion, or increased surface area.

In example assembly A above, heat transfer enhancement by impingement may refer to flow of fluid passing through the outer tube, whose average direction as it proceeds from inlet to outlet may be substantially parallel to the inner tube, being purposely directed to flow toward the inner tube, for example, using geometric features introduced into the annulus. In some embodiments, this directed (impinging) flow may, for example, be perpendicular to the inner tube, or directed toward the inner tube at an angle greater than thirty degrees relative to an axis of the inner tube, while its velocity may be relatively greater than a superficial velocity of the outer-tube fluid (for example, the volumetric flow of the outer-tube fluid divided by the area of the annular cross-section between the inner and outer tube). In some embodiments, the geometric features that promote impingement may include, for example, nozzles and/or openings oriented toward the inner tube, and/or obstructions placed in a flow path that may redirect fluid from a direction that is more parallel to the inner tube more directly towards the outer surface of the inner tube. These example features may be implemented in a periodic fashion, for example, resulting in impingement zones that occur at intervals along the length and/or the circumference of the inner tube. Applicant has discovered that introduction of such impingement features may increase the rate of heat transfer relative to the rate that would be attained by parallel flow through the outer tube. Moreover, Applicant has discovered that for a suitable level of heat transfer enhancement, a ratio of a velocity of an impinging flow to the superficial velocity may be greater than two, greater than five, or greater than ten. The velocity of the impinging flow may be approximated, in the case of nozzles or openings, as the volumetric flow divided by the total flow area defined by the nozzles or openings, through which the flow is directed. Additionally, the heat transfer enhancement may be found to be more suitable at distances between the impingement-inducing feature (e.g., between nozzles or openings 54) and the inner tube that are from about the diameter to about twelve times the diameter of the nozzles or openings 54, or from about the diameter to about ten times the diameter, or from about two times the diameter to about eight times the diameter. Examples of impingement features may include plate impingement and/or piccolo impingement.

The example assembly A above, wherein the outer tube includes a plate impingement disposed between an upstream end and a downstream end. The plate impingement may include a first channel having a stage inlet at the upstream end being closed to flow at the downstream end. The plate impingement also may include a second channel having a stage outlet at the downstream end. The second channel may be disposed between the first channel and the inner tube. The plate impingement may further include a wall separating the first channel from the second channel. The wall may define openings to fluidly connect the first channel and the second channel. The plate impingement may be configured to receive feed through the stage inlet, flow feed from the first channel to the second channel via the openings of the wall to impinge onto an outer surface of the inner tube, and exhaust feed through the stage outlet of the second channel.

The example assembly A above, wherein the outer tube includes a piccolo impingement. The piccolo impingement may include an upstream divider disposed about the inner tube and within the outer tube, a downstream divider disposed about the inner tube and within the outer tube downstream of the upstream divider within annulus. The downstream divider may define at least one stage outlet. The piccolo impingement also may include a chamber defined within the outer tube and about the inner tube between the upstream divider and the downstream divider, and piccolo tubes parallel to or at an angle with respect to the inner tube, and/or may be straight, curved, or bent, and/or offset from the inner tube. The piccolo tubes may extend through the chamber from the upstream divider to the downstream divider. The piccolo tubes may include a stage inlet for receiving incoming feed, and a plurality of openings defined therein, and the openings may be oriented toward the outer surface of the inner tube. The piccolo impingement may be configured to receive feed from the stage inlets, flow feed from the piccolo tubes into the chamber via the plurality of openings, impinge the flow onto the outer surface of the inner tube, and/or exhaust feed from the chamber via the at least one stage outlet.

The example assembly A above, wherein: the at least one stage includes a first stage and a second stage; and one or more of: (1) the inner tube of the first stage has a first outlet and is configured to flow hot reactor effluent from the first inlet to the first outlet, the outer tube of the first stage has a second outlet and is configured to flow feed from the second inlet to the second outlet, and the second inlet of the first stage is adjacent the first outlet of the first stage and the second outlet of the first stage is adjacent the first inlet of the first stage; (2) the second stage includes at least one heat transfer enhancement including one or more of a plate impingement, a piccolo impingent, turbulence promotion, or increased surface area, the second stage being in series with the first stage; or (3) the second stage including an inner tube having a first inlet and a first outlet and is configured to flow reactor effluent from the first inlet of the second stage to the first outlet of the second stage; an outer tube having a second inlet and a second outlet and is configured to flow feed from the second inlet of the second stage to the second outlet of the second stage; and the second inlet of the second stage is adjacent the first inlet of the second stage and the second outlet of the second stage is adjacent the first outlet of the second stage.

The example assembly A above, wherein the at least one heat transfer enhancement of the first stage includes first impingement holes having a first diameter and the at least one heat transfer enhancement of the second stage includes second impingement holes having a second diameter, the second diameter being different from the first diameter.

The example assembly A above, wherein the inner tube includes a heat transfer enhancement.

The example assembly A above, wherein the inner tube has a first outlet and configured to flow hot reactor effluent from the first inlet to the first outlet, the outer tube having a second outlet and configured to flow feed from the second inlet to the second outlet, and one of: the second inlet is adjacent the first outlet and the second outlet is adjacent the first inlet; or the first inlet is adjacent second inlet and the first outlet is adjacent the second outlet.

The example assembly A above, wherein the thermal energy recovery assembly includes a plurality of inner tubes parallel with one another with each inner tube disposed within an outer tube, each outer tube having one or more at least one of a plate impingement, a piccolo impingement, or turbulence promotion to enhance heat transfer from the inner tube to an annulus defined within the outer tube. In some embodiments, the thermal energy recovery assembly may include a plurality of inner tubes parallel with one another, with each inner tube disposed within an outer tube, and with the outer tube and optionally the inner tube having one or more heat transfer enhancements to enhance heat transfer from the inner tube to an annulus defined within the outer tube.

The example assembly A above, wherein the thermal energy recovery assembly is configured: to cool the hot reactor effluent at a rate of at least 2.5 degrees Kelvin/millisecond, at least 3.5 degrees Kelvin/millisecond, at least 4.5 degrees Kelvin/millisecond, at least 5 degrees Kelvin/ms, or at least 5.5 degrees Kelvin/ms, for example, where the cooling rate may be defined as the inlet temperature of the hot reactor effluent (in degrees K) minus 923 K, divided by the residence time required for cooling from the temperature of the hot reactor effluent to 923 K, unless the inlet temperature of the hot reactor effluent (in degrees K) is less than 923 K or the temperature of the cooled reactor effluent is greater than 923K, in which case the cooling rate may be defined as the inlet temperature of the hot reactor effluent (in degrees K) minus the temperature of the cooled reactor effluent when it leaves the thermal energy recovery assembly, divided by the residence time of the effluent in the assembly. In some embodiments, the thermal energy recovery assembly may be configured such that a pressure drop of the hot reactor effluent passing through the thermal energy recovery assembly is less than 0.35 bar, less than 0.30 bar, less than 0.25 bar, or less than 0.20 bar; such that a residence time of the hot reactor effluent within the thermal energy recovery assembly is less than 100 milliseconds, less than 95 milliseconds, less than 90 milliseconds, less than 85 milliseconds, is less than 83 milliseconds, or is less than 80 milliseconds; or such that a pressure drop of the feed passing through the thermal energy recovery assembly is less than 15 bar, less than 12 bar, less than 10 bar, less than 8 bar, or less than 6 bar.

The example assembly A above, wherein the thermal energy recovery assembly is configured to preheat the feed to at least 3500 Celsius, at least 3750 Celsius, at least 4000 Celsius, at least 4250 Celsius, at least 4500 Celsius, at least 4750 Celsius, at least 5000 Celsius, at least 5250 Celsius, at least 5500 Celsius, at least 575° Celsius, at least 6000 Celsius, at least 6250 Celsius, or at least 6500 Celsius.

The example assembly A above, wherein the hot reactor effluent enters the thermal energy recovery assembly at a temperature greater than 575° Celsius, greater than 6000 Celsius, greater than 6100 Celsius, greater than 6200 Celsius, greater than 6300 Celsius, greater than 6400 Celsius, or greater than 6500 Celsius.

A furnace assembly for heating a feed to provide a hot reactor effluent may include the example thermal energy recovery assembly A above; and an electrically powered reactor furnace including a reaction zone configured to heat a feed to a cracking temperature thereof of one or more of ethane, propane, butane, condensate, light naphtha, heavy naphtha, gas oil, pyrolysis oil, materials derived from processing refinery streams, Fischer-Tropsch products, plastic waste, or biofeedstocks.

A method B to produce olefins may include supplying a hydrocarbon feed to an outer tube of a thermal energy recovery assembly, and heating the hydrocarbon feed in the outer tube of the thermal energy recovery assembly to output a preheated hydrocarbon feed. The example method B also may include supplying the preheated hydrocarbon feed to an electrically heated cracking furnace comprising a reaction zone to heat the preheated hydrocarbon feed, and cracking the preheated hydrocarbon feed in the reaction zone to output hot reactor effluent comprising cracked hydrocarbons and olefins. The example method B further may include supplying the hot reactor effluent to an inner tube of the thermal energy recovery assembly, and supplying additional hydrocarbon feed to the outer tube of the thermal energy recovery assembly. The example method B also may include heating the additional hydrocarbon feed by transferring heat from the hot reactor effluent to the additional hydrocarbon feed via the thermal energy recovery assembly.

The example method B above, wherein one or more of: (1) supplying the hot reactor effluent to the inner tube of the thermal energy recovery assembly includes quenching the hot reactor effluent via heat transfer to the additional hydrocarbon feed; or (2) heating the hydrocarbon feed in the outer tube of the thermal energy recovery assembly comprises preheating the hydrocarbon feed via heat transfer to the hydrocarbon feed from the hot reactor effluent. The example method B above, further including enhancing the heat transfer to the additional hydrocarbon feed by providing heat transfer enhancements on one or more of the outer tube or the inner tube, the heat transfer enhancements including one or more of plate impingement, piccolo impingement, or turbulence promotion.

The example method B above, wherein supplying the hot reactor effluent to the inner tube of the thermal energy recovery assembly includes supplying the hot reactor effluent to the inner tube of the thermal energy recovery assembly at a temperature of at least 350° Celsius, at least 375° Celsius, at least 400° Celsius, at least 425° Celsius, at least 450° Celsius, at least 475° Celsius, at least 500° Celsius, at least 525° Celsius, at least 550° Celsius, at least 575° Celsius, at least 600° Celsius, at least 625° Celsius, or at least 650° Celsius.

The example method B above, wherein heating the hydrocarbon feed in the outer tube of the thermal energy recovery assembly to output a preheated hydrocarbon feed comprises heating the hydrocarbon feed to a temperature of at least 350° Celsius, at least 375° Celsius, at least 400° Celsius, at least 425° Celsius, at least 450° Celsius, at least 475° Celsius, at least 5000 Celsius, at least 525° Celsius, at least 550° Celsius, at least 575° Celsius, at least 600° Celsius, at least 625° Celsius, or at least 650° Celsius.

1. A method to produce olefins, the method comprising:
at least one cooling step in a thermal energy recovery assembly characterized by:
supplying a hydrocarbon feed to an outer tube of the thermal energy recovery assembly;
heating the hydrocarbon feed in the outer tube of the thermal energy recovery assembly to output a preheated hydrocarbon feed;
supplying the preheated hydrocarbon feed to an electrically powered cracking furnace comprising a reaction zone to heat the preheated hydrocarbon feed;
cracking the preheated hydrocarbon feed in the reaction zone of the electrically heated cracking furnace using heat generated by electricity to output hot reactor effluent comprising cracked hydrocarbons and olefins;
supplying the hot reactor effluent to an inner tube of the thermal energy recovery assembly; and
cooling the hot reactor effluent in the inner tube of the thermal energy recovery assembly by transferring heat to the hydrocarbon feed; and
whereby the inner tube outside temperature in the thermal energy recovery assembly stays below 720° C. and the inner tube inside temperature in the thermal energy recovery assembly remains above 160° C.

2. The method according to claim 1, where the inner tube outside temperature stays below 720° C. for operation with ethane type feedstock, stays below 700° C. for operation with propane type feedstock, stays below 680° C. for operation with butane type feedstock, stays below 660 for operation with naphtha type feedstock, and stays below 640° C. for operation with feedstocks heavier than naphtha.

3. The method according to claim 1, where the inner tube inside temperature stays above 160° C. for operation with ethane type feedstock, stays above 170° C. for operation with propane type feedstock, stays above 180° C. for operation with butane type feedstock, and stays above 270° C. for operation with naphtha type or heavier feedstocks.

4. The method according to claim 1, where the hot reactor effluent is first partially cooled against the hydrocarbon feed through a co-current section and then further cooled against the hydrocarbon feed in a countercurrent section.

5. The method according to claim 1, where the hot reactor effluent is first cooled against the hydrocarbon feed through a co-current section, further cooled through a steam generating section, and then further cooled against the hydrocarbon feed in a counter-current section

6. The method according to claim 4, wherein the hydrocarbon feed passes first through the co-current section, and then through the countercurrent section.

7. The method according to claim 4, wherein the hydrocarbon feed passes first through the countercurrent section, and then through the co-current section.

8. The method according to claim 4, whereby the countercurrent section is a separate piece of equipment and is a tube-in-tube or shell-and-tube type heat exchanger.

9. A thermal energy recovery assembly for operating the method according to claim 1, the thermal energy recovery assembly comprising:
an outer tube; and
an inner tube;
wherein a hydrocarbon feed is supplied to an electrically powered cracking furnace comprising a reaction zone to heat the hydrocarbon feed;
wherein the inner tube comprises a first inlet configured to receive the hot reactor effluent from the electrically powered cracking furnace; and
wherein the outer tube is disposed about the inner tube to enclose an annulus about the inner tube, the annulus comprising a second inlet configured to receive the hydrocarbon feed.

10. The thermal energy recovery assembly according to claim 9, wherein the annulus comprises at least one heat transfer enhancement to enhance heat transfer from the inner tube to the annulus.

11. The thermal energy recovery assembly according to claim 10, wherein at least one heat transfer enhancement comprises one or more of impingement, turbulence promotion, high-shear-inducing geometry, or increased surface area.

12. The thermal energy recovery assembly according to claim 11, wherein the annulus comprises a plate impingement between an upstream end and a downstream end, the plate impingement comprising:
a first channel having a stage inlet at the upstream end and being closed to flow at the downstream end;
a second channel having a stage outlet at the downstream end, the second channel being disposed between the first channel and the inner tube;
a wall separating the first channel from the second channel, the wall defining openings to fluidly connect the first channel and the second channel; and
the plate impingement being configured to receive feed through the stage inlet, flow feed from the first channel to the second channel via the openings of the wall, and causing flow of the feed to impinge onto an outer surface of the inner tube and exhaust feed through the stage outlet of the second channel.

13. The thermal energy recovery assembly according to claim 11, wherein the annulus comprises a piccolo impingement, the piccolo impingement comprising:
an upstream divider disposed about the inner tube and within the outer tube;
a downstream divider disposed about the inner tube and within the outer tube downstream of the upstream divider within the annulus, the downstream divider defining at least one stage outlet;
a chamber defined within the outer tube and about the inner tube between the upstream divider and the downstream divider; and
piccolo tubes offset from the inner tube, the piccolo tubes extending through the chamber from the upstream divider to the downstream divider, the piccolo tubes including a stage inlet for receiving incoming feed, the piccolo tubes including a plurality of openings defined therein, the piccolo impingement being configured to receive feed from the stage inlets, flow feed from the piccolo tubes into the chamber via the plurality of openings, and exhaust feed from the chamber via the at least one stage outlet.

14. The thermal energy recovery assembly according to claim 9, wherein the thermal energy recovery assembly comprises a plurality of inner tubes parallel with one another with each inner tube disposed within an outer tube, each outer tube having one or more of impingement, turbulence promotion, high-shear-inducing geometry, or increased surface area to enhance heat transfer from the inner tube to an annulus defined within the outer tube.

15. Use of a thermal energy recovery assembly according to claim 9 for conducting a method to produce olefins, the method comprising:
at least one cooling step in a thermal energy recovery assembly characterized by:
supplying a hydrocarbon feed to an outer tube of the thermal energy recovery assembly;
heating the hydrocarbon feed in the outer tube of the thermal energy recovery assembly to output a preheated hydrocarbon feed;
supplying the preheated hydrocarbon feed to an electrically powered cracking furnace comprising a reaction zone to heat the preheated hydrocarbon feed;
cracking the preheated hydrocarbon feed in the reaction zone of the electrically heated cracking furnace using heat generated by electricity to output hot reactor effluent comprising cracked hydrocarbons and olefins;
supplying the hot reactor effluent to an inner tube of the thermal energy recovery assembly; and
cooling the hot reactor effluent in the inner tube of the thermal energy recovery assembly by transferring heat to the hydrocarbon feed; and
whereby the inner tube outside temperature in the thermal energy recovery assembly stays below 720° C. and the inner tube inside temperature in the thermal energy recovery assembly remains above 160° C.

16. The method according to claim 5, wherein the hydrocarbon feed passes first through the co-current section, and then through the countercurrent section.

17. The method according to claim 5, wherein the hydrocarbon feed passes first through the countercurrent section, and then through the co-current section.

18. The method according to claim 5, whereby the countercurrent section is a separate piece of equipment and is a tube-in-tube or shell-and-tube type heat exchanger.
